(19) United States
(12) Patent Application Publication
(10) Pub. No.: US 2026/0288735 A1
(43) Pub. Date: Sep. 24, 2026

(54) SYSTEM

(71) Applicant: SoftBank Group Corp., Tokyo (JP)

(72) Inventor: Takuya FUJITA, Tokyo (JP)

(73) Assignee: SoftBank Group Corp., Tokyo (JP)

(21) Appl. No.: 19/567,014

(22) Filed: Mar. 14, 2026

(30) Foreign Application Priority Data
Mar. 19, 2025 (JP) 2025-045060

(51) Int. Cl. G06F 16/2457; G06F 16/242; G06F 16/28; G16H 20/60

(52) U.S. Cl. CPC G06F 16/24578; G06F 16/243; G06F 16/285; G16H 20/60

(57) ABSTRACT

A system includes a processor that is configured to record a user's meal content as image data, identify food ingredients by using an image analysis technique on the image data, and estimate nutrients based on the identified food ingredients, record a user's amount of exercise by using an accelerometer and a location information technique, and calculate energy expenditure based on recorded data, and evaluate a nutritional status of the user by using recorded data of the meal content and the amount of exercise, and create a prompt for instructing a generative AI model to recommend specific nutritional supplements or protein products.

### CROSS-REFERENCE TO RELATED APPLICATION

This application is based on and claims priority under 35 USC 119 from Japanese Patent Application No. 2025-045060 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional health management and diet support systems generally rely on manual input by users for recording meal content and exercise, which is time-consuming, error-prone, and often incomplete. As a result, accurate estimation of nutritional intake and energy expenditure is difficult, and the generated recommendations tend to be generic and not fully tailored to the user's actual daily behavior. Furthermore, existing systems typically do not integrate image-based food recognition with sensor-based exercise measurement, and therefore fail to comprehensively evaluate a user's nutritional status. In addition, conventional recommendation engines do not effectively exploit generative AI models through structured prompts, and thus cannot dynamically generate personalized suggestions for nutritional supplements, protein products, or meal menus. These systems also rarely take into account the user's emotional state when generating recommendations, and they do not deliver advice in an interactive conversational format that encourages sustained user engagement.

### SUMMARY

In order to solve the above-mentioned problems, the present invention provides a system comprising a processor configured to record a user's meal content as image data, identify food ingredients by using an image analysis technique on the image data, and estimate nutrients based on the identified food ingredients. The processor is further configured to record a user's amount of exercise by using an accelerometer and a location information technique, and to calculate energy expenditure based on the recorded data. The processor is configured to evaluate a nutritional status of the user by using the recorded data of the meal content and the amount of exercise, and to create a prompt for instructing a generative AI model to recommend specific nutritional supplements or protein products. In certain embodiments, the processor is configured to create a prompt, based on a nutritional deficiency status and an emotional state of the user, for instructing the generative AI model to generate recommendations of specific nutritional supplements or meal menus. In addition, the processor is configured to transmit generated advice to the user in a conversational format by using natural language processing technology, thereby enabling interactive and personalized communication that reflects the user's dietary behavior, exercise patterns, nutritional status, and emotional condition.

The term “processor” refers to any hardware and/or software based computation unit, including but not limited to a central processing unit (CPU), graphics processing unit (GPU), microcontroller, dedicated logic circuitry, or a combination thereof, that executes instructions to perform the functions described in the present invention.

The term “user” refers to an individual whose meal content, exercise amount, nutritional status, emotional state, or related data are recorded, analyzed, or for whom recommendations and advice are generated by the system.

The term “meal content” refers to the food and beverage items consumed by the user, including types of foods, ingredients, portion sizes, and any other characteristics relevant to nutritional analysis.

The term “image data” refers to digital image information acquired by an image capturing device, such as a camera integrated into a smartphone or other terminal, that visually represents the user's meal content.

The term “image analysis technique” refers to any computational method for processing image data, including but not limited to image recognition, object detection, segmentation, or classification, used to identify food ingredients or meal components in the image data.

The term “food ingredients” refers to individual food items or components present in the user's meal content, such as specific dishes, raw ingredients, or prepared foods, which are identifiable from image data and usable for nutritional estimation.

The term “nutrients” refers to nutritional components associated with food ingredients, including energy (calories), macronutrients (such as protein, fat, and carbohydrates), micronutrients (such as vitamins and minerals), and other dietary substances relevant to nutritional evaluation.

The term “accelerometer” refers to a sensor that measures acceleration forces acting on the terminal, and that is used to detect movement or physical activity of the user for the purpose of recording the amount of exercise.

The term “location information technique” refers to any technology for acquiring positional data of the terminal, such as Global Positioning System (GPS), assisted GPS, Wi-Fi positioning, or cellular network positioning, used to measure movement, distance, or speed of the user.

The term “amount of exercise” refers to quantitative information related to the user's physical activity, including but not limited to duration, distance, speed, steps, or intensity, which is derived from data acquired by the accelerometer and the location information technique.

The term “energy expenditure” refers to the estimated amount of energy, typically expressed in calories or kilocalories, that the user consumes as a result of physical activity, and that is calculated based on the recorded exercise data and, optionally, user profile information.

The term “nutritional status” refers to an assessment of the user's nutritional condition, including sufficiency or deficiency of energy and specific nutrients such as protein or vitamins, derived from recorded meal content and exercise data.

The term “generative AI model” refers to an artificial intelligence model capable of generating content, such as text, recommendations, or messages, in response to input data or instructions, including but not limited to large language models and other generative machine learning models.

The term “prompt” refers to a structured set of instructions, parameters, or input text generated by the processor and provided to the generative AI model, which specifies a task for the model to perform, such as recommending specific nutritional supplements, protein products, or meal menus.

The term “nutritional supplements” refers to products intended to supplement the user's diet, including but not limited to vitamins, minerals, amino acids, herbal products, and other dietary supplements, which can be recommended to address nutritional deficiencies.

The term “protein products” refers to foods or supplements in which protein is a principal component, including but not limited to protein powders, shakes, bars, or high-protein foods, which can be recommended in relation to the user's exercise and protein intake.

The term “meal menus” refers to proposed combinations of meals, dishes, or recipes, including specific foods and portion sizes, designed to meet nutritional objectives or address identified deficiencies in the user's diet.

The term “nutritional deficiency status” refers to information indicating that one or more nutrients, such as protein, vitamins, or minerals, are estimated to be insufficient for the user over a given period, based on recorded meal content and exercise data.

The term “emotional state” refers to a psychological or affective condition of the user, such as stress, fatigue, motivation, or mood, that is inferred or recorded and is used as a factor in tailoring recommendations or advice.

The term “generated advice” refers to guidance, recommendations, or explanatory information produced based on the user's nutritional status, exercise data, emotional state, and outputs of the generative AI model, and intended to support the user's health or dietary behavior.

The term “natural language processing technology” refers to computational techniques for analyzing, understanding, generating, or transforming human language text, which enable the system to produce and present advice in a human-readable conversational format.

The term “conversational format” refers to a style of interaction in which information, advice, or recommendations are exchanged between the system and the user as a series of messages resembling a dialogue, such as in a chat interface or similar communication channel.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

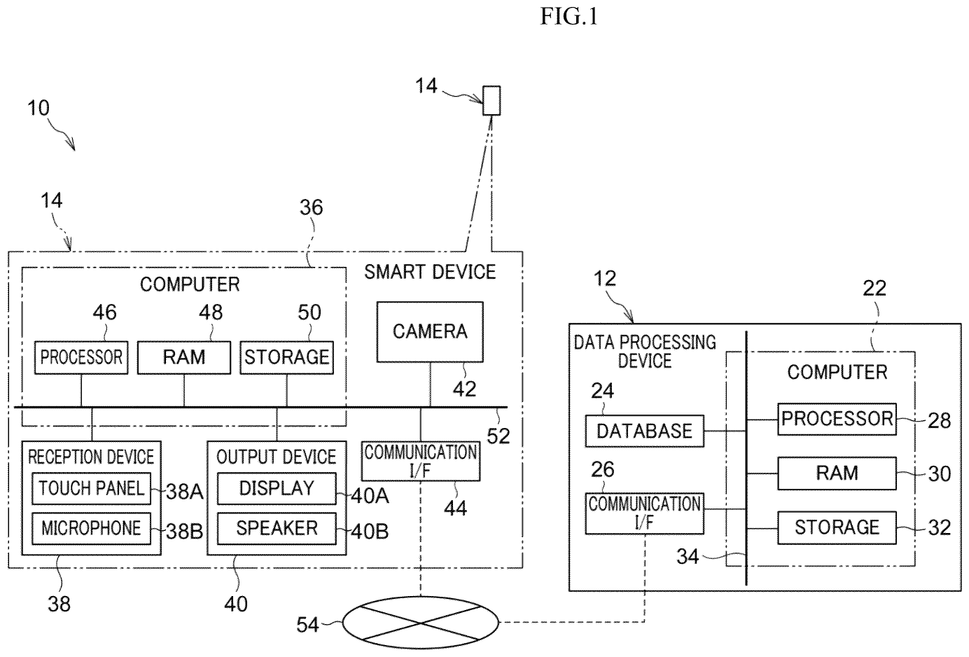

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

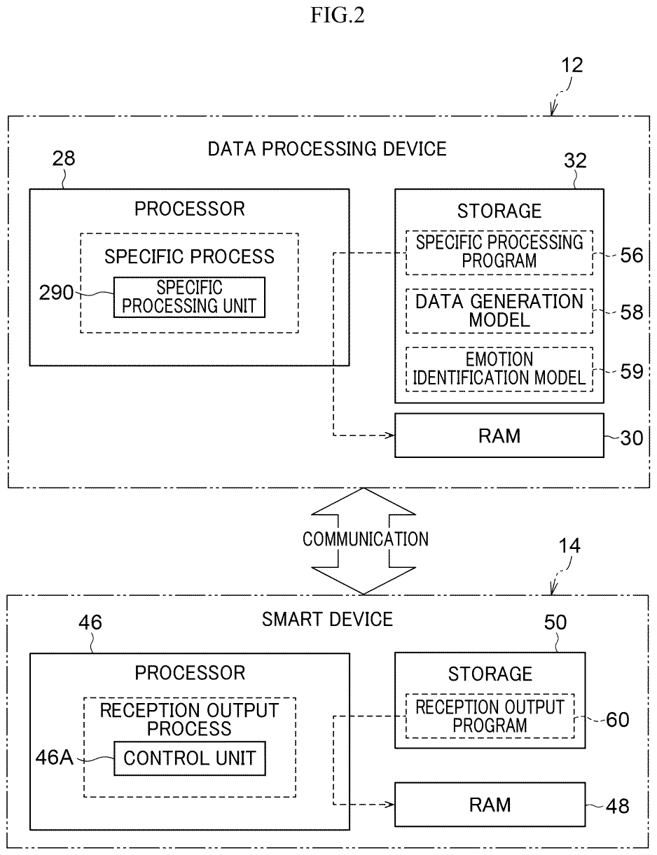

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented health management systems mainly function as passive loggers of meal information and exercise information. In many cases, such systems merely store manually entered numeric values or simple text notes, and then perform fixed-rule calculations such as summing calories or counting steps. As a result, the underlying computer resources, including processors, storage devices, and communication interfaces, are used in a simplistic manner that does not fully exploit contextual data such as images of meals, detailed time-series motion data, or user-specific attributes. This leads to several technical problems from the perspective of computer technology.

First, when meal information is handled only as coarse text or manually entered numeric values, the computation units of a server cannot automatically extract structured, nutrition-related data from rich image information. Image data remains largely unstructured binary content that is not effectively transformed into machine-usable features. Consequently, downstream processes, including analysis and recommendation generation, are limited to low-granularity information, and the system cannot efficiently leverage the available sensor and image-processing capabilities of the computing environment.

Second, in many existing systems, exercise information is stored as simple aggregate values such as daily step counts without preserving the underlying time-series sensor data and location information. This prevents the processor from performing fine-grained computation, such as precise movement distance calculation, dynamic energy expenditure estimation, or correlation of exercise patterns with meal timing. Accordingly, the processing pipeline from sensor input to stored exercise record information is not optimized to utilize motion detection devices and position specifying techniques in a manner that maximizes the usefulness of the collected data for later analysis.

Third, conventional systems that attempt to provide personalized advice often rely on static rule sets or templates that are hard-coded into application logic. When such systems are extended to use a generative AI model, the interaction is frequently limited to generic natural language prompts that are not tightly coupled with the structured health-related data stored in the server. As a result, the server does not systematically construct prompt sentences that incorporate a health status index, nutrition-related information, exercise record information, and user attribute information in a unified, machine-optimized format. This reduces the quality and consistency of responses from the generative AI model and prevents effective reuse of structured health data across multiple inference cycles. Fourth, existing architectures tend to treat evaluation information and improvement proposal information generated by an AI model as unstructured text that is simply displayed to the user. There is no standardized mechanism to parse, organize, and store such information as structured health evaluation results and behavioral guideline information that can then be reused by other components of the system, such as feedback modules, history analysis modules, or dialogue engines. This limits the system's ability to support iterative, context-aware guidance and to implement efficient dialogue-type interactions based on accumulated AI-generated content.

Accordingly, there is a need for a computer-implemented technique that improves the functioning of a server and associated processors by: (i) transforming heterogeneous sensor data and image information into structured nutrition-related information and exercise record information; (ii) aggregating such information to generate a health status index; (iii) constructing and dynamically updating prompt sentences for a generative AI model using the structured data; and (iv) converting AI outputs into reusable, structured health evaluation results and behavioral guideline information that can drive interactive, dialogue-type user interfaces. Such a technique would improve the way computers store, process, and utilize health-related data and AI-generated information, thereby improving the overall operation of the computer system itself.

The specific processing by the specific processing unit 290 of the data processing device 12 in Example 1 is realized by the following means.

The present invention provides a server comprising a processor and a storage device, the processor being configured to acquire meal information of a user as image information and character information, analyze the image information to classify intake objects, calculate nutrition-related information on the basis of a classification result of the intake objects and the character information, acquire exercise amount information of the user as time-series data by using a motion detection device and a position specifying technique, calculate a movement distance, a step count, and an energy consumption on the basis of the time-series data, store exercise record information, store the nutrition-related information and the exercise record information in the storage device, aggregate a nutrition intake amount and an exercise amount over a predetermined period, generate a health status index, construct a prompt sentence to be used as an input to a generative AI model on the basis of the health status index and attribute information of the user, include the nutrition-related information and the exercise record information in the prompt sentence, transmit the prompt sentence to the generative AI model, analyze evaluation information and improvement proposal information in natural language obtained from the generative AI model, organize and store the evaluation information and the improvement proposal information as a health evaluation result and behavioral guideline information for each user, and deliver the health evaluation result and the behavioral guideline information to a terminal device and present the health evaluation result and the behavioral guideline information to the user by display control. This enables improvement of computer functionality by transforming unstructured sensor data and image information into structured health-related data, by programmatically constructing and updating prompt sentences that tightly couple such structured data with a generative AI model, and by converting AI outputs into reusable, structured evaluation and guideline information, thereby allowing the server to perform more efficient data storage, retrieval, and interactive presentation operations than conventional systems.

The term “meal information” refers to information indicating food and drink intake of a user, including at least one of image information representing a meal and character information representing names, types, amounts, or attributes of intake objects.

The term “image information” refers to digital data representing a still image or moving image of a meal or food item, obtained by an image capturing device and processable by image analysis techniques.

The term “character information” refers to text data including characters, numerals, symbols, or codes that describe meal content, intake objects, quantities, times, or related attributes, and that are input by a user or generated by a computer.

The term “intake objects” refers to edible or drinkable items, including foods, beverages, or ingredients, that are consumed by a user and are subject to classification and nutrition estimation.

The term “nutrition-related information” refers to data indicating nutritional properties of intake objects, including at least one of energy amount, macronutrient content, micronutrient content, or other dietary attributes, which is calculated on the basis of meal information.

The term “exercise amount information” refers to data indicating physical activity of a user, including time-series sensor values, movement patterns, or derived metrics such as step count, distance, or energy expenditure.

The term “time-series data” refers to data composed of multiple values associated with respective time points or time intervals, obtained sequentially from a sensor or position specifying technique during a period of user activity.

The term “motion detection device” refers to a hardware sensor or sensor assembly, such as an acceleration sensor or inertial measurement unit, that detects physical movement of a terminal or a body part and outputs motion-related signals.

The term “position specifying technique” refers to a technique for determining geographic position, including at least one of satellite-based positioning, network-based positioning, or sensor-based positioning, and providing location coordinates or related positional data.

The term “movement distance” refers to a value representing a length of a path traveled by a user during a period of exercise, which is calculated on the basis of position-related time-series data.

The term “step count” refers to a numerical value indicating a number of steps taken by a user during a period of walking, running, or similar movement, which is estimated on the basis of motion detection data.

The term “energy consumption” refers to a value representing an estimated amount of energy expended by a user during physical activity, calculated using at least one of time duration, movement distance, step count, user attribute information, or activity type.

The term “exercise record information” refers to structured data representing results of processing exercise amount information, including at least one of movement distance, step count, energy consumption, start time, end time, and exercise type.

The term “storage device” refers to a hardware or virtual component capable of storing data, including at least one of a memory device, a magnetic storage unit, a semiconductor storage unit, or a network-accessible storage system.

The term “nutrition intake amount” refers to an aggregated quantity of nutritional elements ingested by a user over a predetermined period, calculated on the basis of nutrition-related information associated with multiple meals.

The term “exercise amount” refers to an aggregated quantity indicating physical activity of a user over a predetermined period, including at least one of total movement distance, total step count, or total energy consumption.

The term “health status index” refers to one or more computed indicators representing a health-related state of a user, derived from a combination of nutrition intake amount, exercise amount, and optionally user attribute information.

The term “attribute information of the user” refers to data describing personal characteristics of a user, including at least one of age, sex, body weight, body height, lifestyle pattern, or health goal, which is used in computation or personalization.

The term “prompt sentence” refers to text data, optionally including structured data representations, that is constructed for input to a generative AI model and that specifies context, data, and instructions to control output generation.

The term “generative AI model” refers to a machine-learned model configured to generate natural language or other content in response to an input prompt sentence, based on parameters trained from data.

The term “evaluation information” refers to natural language content generated by the generative AI model that describes an assessment of a user's health status, nutrition status, exercise status, or related conditions.

The term “improvement proposal information” refers to natural language content generated by the generative AI model that provides recommendations, advice, or suggested actions for improving or maintaining the user's health status.

The term “health evaluation result” refers to structured information derived from evaluation information, representing an organized assessment of a user's health status that is associated with specific time periods or conditions.

The term “behavioral guideline information” refers to structured information derived from improvement proposal information, representing organized instructions or plans for user behavior, including dietary actions or exercise actions.

The term “terminal device” refers to an information processing apparatus used by a user, including at least one of a mobile device, a wearable device, a tablet device, or a computer device, capable of capturing data, communicating with a server, and displaying information.

The term “display control” refers to processing for determining, generating, and updating visual or audio output on a terminal device, in order to present health evaluation results, behavioral guideline information, or dialogue-type responses to a user.

In one or more embodiments, a server, a terminal, and a generative AI model cooperate to implement a health management system that acquires meal information and exercise information of a user, converts heterogeneous sensor data into structured data, constructs prompt sentences for a generative AI model, and returns structured evaluation and guideline information to the terminal.

### A. Overall Hardware and Software Configuration

A server comprises at least one processor, a main memory, a non-volatile storage device, a network interface, and one or more databases. The server executes software components including an application server module, a data processing module, a model interface module for a generative AI model, and a presentation management module. The server may run on a general-purpose computing platform provided by a cloud infrastructure, such as a virtual machine instance, but is not limited thereto.

A terminal comprises at least one processor, a memory, a camera, a motion detection device such as an acceleration sensor, a positioning module that supports a position specifying technique (for example, satellite-based positioning), a display, and a wireless communication module. The terminal executes a health management application that interacts with the server via a network such as a wireless local area network or a mobile communication network. The terminal may be a smartphone, a tablet device, or a wearable device.

The server uses a database management system to store meal information, exercise record information, nutrition-related information, health status indices, evaluation information, and behavioral guideline information. The server uses a data processing library to perform numerical calculations and data transformations. The server uses a model interface library to send prompt sentences to and receive responses from a generative AI model.

The generative AI model is implemented as a neural network model, such as a transformer-based language model. The generative AI model comprises an embedding layer, a plurality of attention blocks, feedforward layers, and an output layer. The generative AI model is trained in advance using a training dataset containing natural language texts, including texts related to nutrition, exercise, and health, but the generative AI model is further adapted to the health domain by fine-tuning on health-specific training data. The server accesses the generative AI model via an application programming interface provided on a separate computing resource or within the same server environment.

### B. Data Structures and Representations

The server defines a meal record as a data structure including at least a user identifier, a timestamp, an image path or image identifier, and character information describing intake objects and quantities. The image information is stored in a binary storage or object storage, while a reference path or identifier is stored in a relational table.

The server defines nutrition-related information as a data structure containing energy values, macronutrient values, micronutrient values, and optionally food category labels.

The server derives these values by linking intake objects to entries in a food composition table.

The server defines exercise record information as a data structure including a user identifier, an exercise type, a start time, an end time, a movement distance, a step count, and an energy consumption value. The server also stores metadata such as sampling rate of the motion detection device and quality indicators of the position specifying technique.

The server defines a health status index as a structure that aggregates nutrition-related information and exercise record information for a predetermined period, such as a day or a week. The health status index may include a calorie balance value, activity level categories, and deviation amounts from target ranges for nutrients and exercise.

The server defines a prompt sentence as a string of characters containing both human-readable natural language and embedded structured information formatted in a consistent pattern. The prompt sentence includes an instruction segment, a data description segment, and a required output segment.

The server defines evaluation information and improvement proposal information as text responses from the generative AI model that are further parsed into a health evaluation result and behavioral guideline information. These parsed structures reference the original health status index for traceability.

### C. Operation of the Terminal

The terminal captures image information of a meal by using the camera. The terminal converts raw image signals into digital data, such as a compressed image file. The terminal associates the digital image file with character information that is entered by the user via a graphical user interface.

The terminal obtains exercise amount information by using the motion detection device and the position specifying technique. The terminal collects time-series acceleration data and location data at predetermined intervals and stores them temporarily in memory. The terminal pre-processes the raw time-series data to reduce noise, for example, by applying a digital filter to acceleration signals.

The terminal calculates a preliminary step count by applying a step-detection algorithm to the filtered acceleration data. The algorithm detects peaks that exceed a dynamic threshold that is adjusted based on the user's movement pattern. The terminal calculates a preliminary movement distance by integrating location differences computed from sequential position samples.

The terminal estimates energy consumption using a parameterized formula that takes as inputs the exercise type, duration, movement distance, step count, and user attribute information such as body weight. The terminal transmits meal information and exercise record information to the server through an encrypted communication channel.

### D. Operation of the Server: Image Analysis and Nutrition Estimation

The server receives image information and character information from the terminal and stores them. The server uses an image analysis engine implemented by a convolutional neural network or a transformer-based vision model to classify intake objects. The image analysis engine is configured with multiple layers of filters that detect edges, textures, and shapes corresponding to typical food items. The server uses this engine to output probability distributions over candidate food categories.

The server combines the classification result from the image analysis engine with character information supplied by the user. The server performs consistency checking by comparing the recognized categories with text labels. If the probability of a category is below a threshold or inconsistent with the character information, the server applies a correction rule or requests an updated label from the terminal in a later synchronization.

The server retrieves nutrition-related base values for each identified intake object from a food composition table stored in the database. The server multiplies base nutrient values per unit mass or volume by the quantities indicated in the character information. The server sums values across all intake objects in a meal to compute nutrition-related information for the meal. The server writes the computed values into a nutrition table referenced by the user identifier and timestamp.

By automating classification of intake objects and mapping to a structured nutrition-related representation, the server reduces manual entry errors and improves the accuracy and granularity of nutrition calculations compared to systems that store only textual or numeric summaries.

### E. Operation of the Server: Exercise Processing and Aggregation

The server receives exercise record information from the terminal. The server optionally re-computes movement distance and energy consumption using higher-precision algorithms and additional contextual data, such as elevation information or temperature, which may be stored as auxiliary data.

The server aggregates exercise record information over a predetermined period by summing movement distance, step count, and energy consumption and by detecting patterns such as continuous streaks of active days. The server stores aggregate values and derived features as part of the health status index. The server uses standardized data structures for exercise and nutrition, which allows efficient indexing and retrieval in the database, decreasing query time and enabling faster responses for subsequent analysis.

### F. Construction of Prompt Sentences and Interaction with the Generative AI Model

The server constructs a prompt sentence on the basis of the health status index and attribute information of the user. The server uses a prompt-construction module that assembles a text string in a pre-defined template. The template may be stored as a parameterized pattern in memory.

The server integrates the following elements into the prompt sentence:
(1) A role specification indicating that the generative AI model should behave as a health coach or advisor.
(2) A description of the health status index including calorie intake, nutrient distribution, exercise amounts, and deviations from targets.
(3) A concise description of user attribute information such as age, body mass category, and primary health goals.
(4) Explicit instructions regarding the desired output format and constraints, such as number of suggestions and length limits.

For example, the server may construct a prompt sentence as follows:

“You are a health coach. Analyze the following daily data and give a brief evaluation and 3 specific suggestions.

Data:
Total energy intake: 1,600 kcal.
Protein: 60 g, Fat: 40 g, Carbohydrates: 250 g.
Exercise: walking 30 minutes, 3,000 steps, 2.5 km, 120 kcal burned.

User goal: maintain a healthy weight and improve cardiovascular fitness.

Output format: first a 3-sentence evaluation, then a numbered list of 3 suggestions.”

In another example, the server may construct a prompt sentence as follows:

“The user ate a low-calorie lunch (about 80 kcal from salad: lettuce 100 g, tomato 50 g) and walked 3,000 steps (2.5 km, 120 kcal burned). Generate practical advice for the rest of the day, including what to eat for dinner to balance nutrients and how much additional exercise would be reasonable. Keep the answer under 200 words.”

The server sends the prompt sentence to the generative AI model via the model interface module. The server sets model parameters such as temperature, top-k, and maximum token count to control variability and length. The generative AI model processes the prompt sentence by encoding the text into token embeddings, applying multiple layers of attention and feedforward transformations, and generating a token sequence that forms evaluation information and improvement proposal information.

### G. Parsing and Structuring of AI Output

The server receives natural language output from the generative AI model. The server does not simply store the output as free text. Instead, the server applies a parsing module that uses rule-based pattern matching and, optionally, a secondary natural language understanding model to divide the output into segments such as overall evaluation, identified problems, and specific recommendations.

The server maps these segments into the health evaluation result and behavioral guideline information structures. For example, the server may convert a sentence indicating “protein intake is slightly low” into a categorical flag and a numerical deviation value associated with the protein nutrient dimension. The server stores this structured representation in the database along with a reference to the original text.

By converting generative outputs into structured data, the server allows efficient querying, aggregation, and longitudinal analysis across multiple days, which is technically different from conventional systems that treat AI-generated advice as unstructured text. This structuring enables search operations using indices and improves response time when generating future prompt sentences that reference past feedback.

### H. Dialogue-Type Interaction and Iterative Refinement

The terminal displays the health evaluation result and behavioral guideline information to the user using display control. The terminal presents both a summarized view and detailed text segments. The user can input additional questions or clarifications via a dialogue interface.

The server treats user follow-up input as an additional context for constructing new prompt sentences. The server includes previously stored health evaluation results and behavioral guideline information as well as the new user question in the next prompt sentence. By referencing structured historical data, the server avoids re-sending all raw data to the generative AI model, which reduces communication load and improves overall latency.

The server updates its internal representations based on subsequent outputs of the generative AI model. The server maintains a dialogue state that includes references to past prompt sentences and responses, which allows non-linear navigation in the dialogue without re-computation of basic statistics. This dialogue state is stored in a data structure optimized for quick retrieval, such as a keyed document store.

### I. Technical Effects and Improvement of Computer Technology

The server improves computer technology in several ways. First, the server transforms raw image information and sensor time-series data, which are large and unstructured, into compact structured representations (nutrition-related information, exercise record information, and health status indices). This transformation reduces storage redundancy and allows the server to perform computations on aggregated features rather than repeatedly processing raw data, which increases processing speed and reduces computational load.

Second, the server dynamically constructs prompt sentences that embed health status indices and user attribute information in a standardized template. This construction ensures that the generative AI model receives well-structured and complete context, resulting in more consistent outputs and reducing the need for multiple retries. By specifying output formats and constraints explicitly, the server obtains responses that are easier to parse and structure, which reduces downstream parsing complexity and error rates.

Third, the server uses a generative AI model that is trained and fine-tuned according to a specific loss function that penalizes inconsistent or incomplete health advice. The training process includes data augmentation such as paraphrasing of health scenarios, simulated noise in numeric inputs, and scenario randomization. The model parameters are updated by gradient descent based on an objective that combines language modeling loss with domain-specific consistency constraints. As a result, the generated evaluation information and improvement proposal information are tailored to the data representations produced by the server's processing pipeline, closing the loop between structured data and AI reasoning.

Fourth, the server's parsing and structuring of AI output create a feedback mechanism where the server can automatically estimate the quality of responses. For example, the server may detect missing essential elements or contradictions between AI suggestions and base health status indices. The server can then adapt prompt sentence templates or modify parameter settings to improve future outputs. This capability is not a mere automation of human reasoning; it is an improvement in how computer systems manage, validate, and reuse AI-generated content.

Fifth, the server's architecture is designed to reduce communication load. Instead of transmitting all raw sensor data to the generative AI model, the server pre-aggregates and encodes the essential features into compact textual summaries in the prompt sentences. This design limits the data size passed to the model interface, which reduces request latency and improves scalability when supporting many users.

Sixth, the server enables improved accuracy in nutrition assessment and exercise evaluation by combining multiple data sources (image information, character information, and sensor data) and by cross-validating them via non-conventional rules, such as comparing estimated portion sizes from image analysis with quantities stated by the user.

The server can flag discrepancies and either correct them or request clarification, reducing systematic errors that would be difficult for a human user to detect manually.

### J. Alternative Embodiments and Variations

In some embodiments, the server deploys the generative AI model locally rather than accessing a remote model. The server may execute the model on a specialized accelerator to further reduce latency. The same data structures and prompt sentence templates can be used in this configuration.

In other embodiments, the terminal performs additional preprocessing, such as on-device classification of food items or initial computation of health status indices for short periods.

The server then refines and integrates these local indices. This offloading strategy further reduces bandwidth requirements and server-side computational load while maintaining consistency through standardized data formats.

In further embodiments, the server adapts prompt sentence content based on device capabilities. For example, if a terminal has limited display space, the server instructs the generative AI model to produce shorter, prioritized behavioral guideline information. This control is realized by adding description segments to the prompt sentence that specify length and priority rules.

In yet other embodiments, the server maintains multiple prompt templates optimized for different objectives, such as short-term motivation, long-term planning, or troubleshooting. The server selects a template based on the health status index and historical user interactions. This multi-template approach constitutes a non-traditional way of interacting with a generative AI model, tailored to the structured data held by the server. Through these embodiments, the server, the terminal, and the generative AI model cooperatively implement a technical solution that goes beyond simple automation of human tasks. The system changes how data is represented, processed, and used for decision support within a computer environment, resulting in improved accuracy, responsiveness, and efficiency in health-related data processing and AI-assisted guidance.

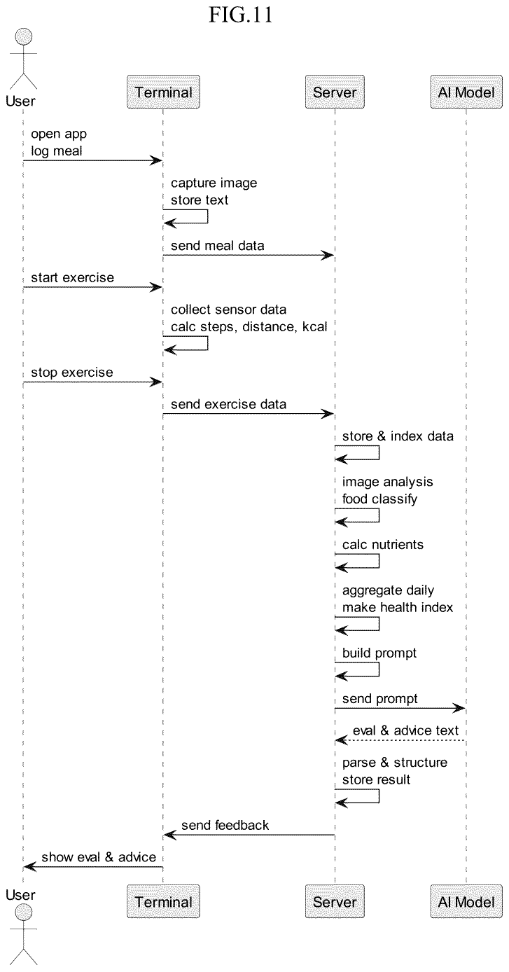

The following describes the processing flow using FIG. 11.

### Step 1:

The user operates the terminal to capture meal image information and input character information.

The user starts the health management application on the terminal, opens a meal logging screen, and uses the camera of the terminal to capture a still image of a meal.

Input: raw optical data from the camera sensor and user keystrokes or touch input specifying food names and quantities.

The terminal converts the raw optical data into digital image data in a compressed format and associates this image data with the entered character information, including intake objects and their amounts.

Output: a meal record candidate containing an image file, character information, a timestamp, and a user identifier in the memory of the terminal.

### Step 2:

The terminal preprocesses and stores meal information in a local storage.

The terminal verifies that the character information includes valid quantities and recognizable text fields, and attaches metadata such as meal type and capture time.

Input: the meal record candidate created in Step 1.

The terminal writes the image file to a local file system or object storage region and stores a structured meal record in a local database, with a reference path to the image and normalized text fields for intake objects and amounts.

Output: a structured meal record stored in the local database of the terminal and ready for transmission to the server.

### Step 3:

The user operates the terminal to start exercise logging.

The user launches an exercise mode screen of the health management application and selects an exercise type, such as walking or running.

Input: user selection of exercise type and the current time.

The terminal records the exercise type and start time and activates a motion detection device and a position specifying technique to begin collecting time-series data.

Output: an active exercise session state in the terminal, including initialized buffers for motion and position data.

### Step 4:

The terminal acquires time-series motion data and position data and computes preliminary exercise metrics.

The terminal repeatedly samples acceleration values from the motion detection device at fixed intervals and obtains location coordinates from the position specifying technique.

Input: raw acceleration samples and raw position coordinates with timestamps.

The terminal applies a digital filter to the acceleration samples to reduce noise, applies a step-detection algorithm to count steps, and calculates movement distance by summing segment distances between sequential position coordinates using a distance formula.

Output: a time-series record of motion and location data, a cumulative step count, and a cumulative movement distance for the current exercise session in the memory of the terminal.

### Step 5:

The terminal estimates energy consumption and finalizes exercise record information.

When the user ends the exercise session, the terminal stops sampling the sensors and calculates the total duration of the activity.

Input: cumulative step count, cumulative movement distance, exercise type, duration, and user attribute information such as body weight stored locally.

The terminal applies an energy estimation formula that combines movement distance, step count, duration, and user attributes to compute energy consumption, and then creates a structured exercise record including all computed values.

Output: a structured exercise record stored in the local database of the terminal.

### Step 6:

The terminal synchronizes meal and exercise records with the server.

The terminal checks network connectivity and, when available, reads unsent meal records and exercise records from the local database.

Input: locally stored structured meal records and exercise records.

The terminal packages the records into a batch payload, converts the records into a structured text or object representation, and transmits the payload to the server via a secure communication channel.

Output: a transmitted data batch containing multiple meal and exercise records received by the server.

### Step 7:

The server stores and indexes received records.

The server validates authentication, parses the incoming batch, and verifies mandatory fields for each record.

Input: a batch of structured meal records and exercise records from the terminal.

The server writes the meal records and exercise records into corresponding tables in a database, creates indices on user identifiers and timestamps, and stores references to associated image files.

Output: persisted meal records and exercise records in the server database, indexed for efficient retrieval.

### Step 8:

The server performs image analysis to classify intake objects.

The server retrieves image references and associated character information for newly received meal records.

Input: image data for meals and character information describing intake objects and quantities.

The server applies an image analysis model, such as a convolutional neural network or a vision transformer, to each image to produce probability scores for candidate food categories, and then merges these scores with the character information using consistency rules.

Output: a list of classified intake objects for each meal record, with confidence scores and corrected labels where needed.

### Step 9:

The server calculates nutrition-related information for each meal.

The server accesses a food composition table to obtain nutrient values per unit amount for each classified intake object.

Input: classified intake objects with quantities and food composition entries.

The server multiplies the unit nutrient values by the corresponding quantities, sums nutrients across all intake objects in the meal, and generates a structured nutrition-related information record that includes energy, macronutrients, and micronutrients.

Output: nutrition-related information records stored in a nutrition table and linked to the original meal records.

### Step 10:

The server aggregates nutrition intake amount and exercise amount over a predetermined period.

The server queries nutrition-related information and exercise record information for a target period, such as one day, for a specific user.

Input: multiple nutrition-related information records and multiple exercise record information records associated with the user and the period.

The server sums energy intake, macronutrient and micronutrient amounts, and exercise metrics such as total movement distance, total step count, and total energy consumption, and then computes derived values such as calorie balance and deviations from target ranges.

Output: a health status index for the user for the specified period, stored in an aggregation table.

### Step 11:

The server constructs a prompt sentence for a generative AI model.

The server retrieves the health status index and attribute information of the user, such as age and health goals.

Input: the health status index and user attribute information.

The server inserts these values into a prompt template by replacing placeholders with actual data, adds instructions describing the role of the generative AI model and the desired output format, and concatenates all parts into a single prompt sentence.

Output: a complete prompt sentence that encodes the user's health status and requested advice in natural language.

### Step 12:

The server sends the prompt sentence to the generative AI model and obtains evaluation information and improvement proposal information.

The server calls an interface of the generative AI model and provides the prompt sentence along with generation parameters such as maximum length and temperature.

Input: the constructed prompt sentence and model parameter settings.

The generative AI model encodes the sentence, applies its internal neural network layers, and generates a sequence of tokens representing an evaluation of the user's health status and specific suggestions; the server receives this sequence and decodes it into natural language text.

Output: evaluation information and improvement proposal information as natural language text.

### Step 13:

The server parses and structures the evaluation information and improvement proposal information.

The server applies a parsing module that detects predefined markers, sentence patterns, or bullet structures in the received text.

Input: evaluation information and improvement proposal information in natural language.

The server separates overall evaluation sentences from recommendation sentences, assigns each recommendation to a category such as nutrition or exercise, and maps them into fields of a health evaluation result and behavioral guideline information structure, which are then stored in the database.

Output: structured health evaluation results and structured behavioral guideline information linked to the health status index and user identifier.

### Step 14:

The server prepares feedback content and transmits it to the terminal.

The server formats the structured health evaluation result and behavioral guideline information into a response payload that includes headings, summaries, and detailed items.

Input: structured health evaluation results and behavioral guideline information.

The server sends the payload to the terminal over the network, optionally triggering a notification to indicate new feedback is available.

Output: a feedback message delivered to the terminal containing evaluation and guideline data.

### Step 15:

The terminal displays the evaluation and guidelines and accepts follow-up input from the user.

The terminal receives the feedback message and updates the health management application's user interface.

Input: the feedback message containing evaluation text, guideline items, and optional formatting hints.

The terminal renders the evaluation information and behavioral guideline information on the display in sections, allows the user to scroll and tap on items for more details, and provides an input field where the user can enter follow-up questions or comments, which the terminal stores for later transmission as additional context for a new prompt sentence.

Output: a visual presentation of current feedback on the terminal and, optionally, new user input that can be used as additional context in subsequent processing.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional health management systems that track dietary intake and physical activity typically rely on manual data entry by the user or on simple rule-based estimations. Such arrangements suffer from several technical problems in terms of computer technology. First, manual entry interfaces increase user burden and lead to incomplete or inaccurate input data, which degrades the quality of data stored in computing resources and reduces the effectiveness of downstream processing. Second, even when image capture or sensor data are used, conventional systems often treat these heterogeneous inputs in isolation, without unified processing pipelines, so that servers must repeatedly execute fragmented computations and database accesses, resulting in inefficient use of processor cycles and memory, and limiting scalability when serving many users in parallel.

Moreover, traditional systems that generate recommendations based on nutritional data tend to use fixed, hard-coded logic or templates. These methods make poor use of modern machine learning resources and fail to adapt outputs to the rich, structured context that can be computed from user data. As a consequence, even when a server maintains large amounts of historical data, the server does not effectively transform such data into machine-usable context for an advanced generative artificial intelligence model. In particular, conventional systems either transmit raw or minimally processed logs to an external recommendation engine, or they provide only coarse summary statistics, both of which constrain the ability of the computing system to generate nuanced, context-sensitive advice.

Additionally, existing architectures typically do not optimize the representation of combined dietary, activity, and emotional-state information as a structured, machine-oriented prompt sentence. Without such optimized prompts, a generative artificial intelligence model consumes more computational resources to infer missing structure, leading to longer inference times, higher network and processor loads, and unstable output quality. Furthermore, known systems do not maintain a coherent dialogue history tightly integrated with structured evaluation data on the server side, and therefore cannot efficiently generate context-aware follow-up advice in a multi-turn conversation while minimizing redundant computation and data transfer.

Accordingly, there is a need for a computer-implemented system that technically improves how dietary image data, sensor-based activity data, user profile data, and emotional-state information are acquired, fused, evaluated, and transformed into structured evaluation data and optimized prompt sentences for a generative artificial intelligence model. There is also a need for a server architecture that reduces user input burden while still obtaining high-quality, machine-usable data, that efficiently aggregates and evaluates nutritional status over time, and that programmatically constructs, updates, and manages prompt sentences and dialogue histories so that generative artificial intelligence resources can be utilized more efficiently and consistently. The present invention addresses these technical problems by providing specific server-side processing flows, data structures, and interactions with user terminals and generative artificial intelligence models.

The specific processing by the specific processing unit 290 of the data processing device 12 in Application Example 1 is realized by the following means.

The present invention provides a server comprising a processor configured to control an imaging device of a user terminal to acquire image data representing food and beverage ingested by a user, execute machine-learning-based image analysis on the image data to identify a type and an amount of the food and beverage, and calculate nutrient component amounts by referencing a nutrition information storage, to control an acceleration detection device and a location information acquisition device of the user terminal to acquire time-series sensor data and movement route data relating to physical activity of the user and to calculate activity type, movement distance, speed, and energy expenditure based on the acquired data, to aggregate intake energy and energy expenditure over a predetermined period, estimate basal metabolism and target energy balance from user profile information, evaluate a nutritional status of the user by comparison and generate structured evaluation data, to summarize the structured evaluation data and associated dietary and physical activity history data into natural-language text including user attributes and the evaluation result, to construct an optimized prompt sentence for a generative artificial intelligence model by appending instructions relating to output format and recommendation content to the text, to transmit the prompt sentence to the generative artificial intelligence model and acquire, store, and return to the user terminal an advice text generated by the model, and, in some embodiments, to further estimate an emotional state of the user from emotional-index information and self-report information, incorporate the emotional state and the structured evaluation data into an additional prompt sentence that instructs the generative artificial intelligence model to generate specific nutritional supplement or meal-menu recommendations, and to maintain dialogue history data and generate follow-up prompt sentences by combining additional user input with the dialogue history and the structured evaluation data for multi-turn, context-aware advice generation. This enables the server to automatically transform heterogeneous sensor and image data into high-quality structured evaluation data and optimized prompt sentences, to reduce user input burden while improving data reliability, to utilize generative artificial intelligence resources more efficiently through machine-structured prompts and server-managed dialogue context, and to provide technically improved, scalable, and responsive health management functionality in a computer-implemented environment.

The term “user terminal” refers to an electronic device operated by a user, including at least one processor, a memory, a display, and one or more input/output interfaces, and configured to acquire sensor data and image data and to communicate with a server over a communication network.

The term “imaging device” refers to an image acquisition component provided in the user terminal, such as a digital camera module or image sensor, that converts an optical image of a subject into digital image data.

The term “image data” refers to digital data representing a still image or a sequence of images, including pixel values and associated metadata, acquired by the imaging device.

The term “food and beverage” refers to any ingestible item, including solid food, liquid drink, and mixed meals, that is consumed by the user and is subject to nutritional analysis.

The term “machine-learning-based image analysis” refers to processing in which the processor applies a trained statistical model, such as a neural network, to image data to infer one or more attributes of objects depicted in the image, including their type or quantity.

The term “type of the food and beverage” refers to a classification label or category, such as a dish name or ingredient class, assigned to the food and beverage by the image analysis processing or by subsequent data mapping.

The term “amount of the food and beverage” refers to a quantitative estimate of a portion of the food and beverage, such as mass, volume, or serving count, derived from the image analysis and related computations.

The term “nutrition information storage” refers to a data storage resource, such as a database or memory, that holds information associating food and beverage types and amounts with corresponding nutritional values.

The term “nutrient component amounts” refers to quantitative values that indicate nutritional content of the food and beverage, including at least energy, macronutrients, and optionally micronutrients, for a given portion.

The term “acceleration detection device” refers to a sensor provided in the user terminal that measures acceleration along one or more axes and outputs time-series acceleration data.

The term “location information acquisition device” refers to a component or combination of components, such as a positioning receiver or communication module, that obtains geographic position information of the user terminal, including at least coordinates and optionally time.

The term “time-series sensor data” refers to a sequence of sensor measurements, such as acceleration values, each associated with a time index, indicating changes of a physical quantity over time.

The term “movement route data” refers to data indicating a trajectory of movement of the user or user terminal over time, including a sequence of position values and corresponding timestamps.

The term “activity type” refers to a classification of a physical activity of the user, such as walking, running, or resting, inferred from the sensor data and movement route data.

The term “movement distance” refers to a calculated value representing a linear distance traveled by the user or user terminal over a given period, based on the movement route data.

The term “speed” refers to a scalar value representing a rate of change of position of the user or user terminal over time, calculated from the movement route data.

The term “energy expenditure” refers to a calculated estimate of caloric energy consumed by the user as a result of physical activity, derived from the sensor data, movement distance, speed, and optionally user profile information.

The term “intake energy” refers to an amount of caloric energy consumed by the user through food and beverage intake, calculated from the nutrient component amounts for one or more meals.

The term “predetermined period” refers to a time interval defined by the system, such as a day, multiple days, or another time window, over which intake energy and energy expenditure are aggregated.

The term “user profile information” refers to stored data associated with the user, including at least age, sex, height, weight, and optionally goals or activity level, used for health-related calculations.

The term “basal metabolism” refers to an estimated amount of energy required to maintain basic life functions of the user at rest, calculated based on the user profile information using a predefined formula.

The term “target energy balance” refers to a desired relationship between intake energy and energy expenditure for the predetermined period, determined based on the user profile information and user goals.

The term “nutritional status” refers to a condition of the user in terms of energy intake, energy expenditure, and nutrient balance, evaluated by comparing computed values with reference values such as basal metabolism and target energy balance.

The term “structured evaluation data” refers to data in a predefined structured format, such as a record or data object, representing results of an evaluation of nutritional status, including at least aggregated numeric values and one or more derived indicators.

The term “dietary history data” refers to stored records of food and beverage intake by the user over time, including at least timestamps, types, amounts, and associated nutritional values.

The term “physical activity history data” refers to stored records of physical activities of the user over time, including at least activity types, time intervals, movement distances, speeds, and energy expenditures.

The term “natural-language text” refers to a sequence of characters or tokens that form one or more sentences in a human language and is interpretable by a human reader.

The term “prompt sentence” refers to a natural-language text or sequence of tokens provided as input to a generative artificial intelligence model, the text including descriptive content and instructions that guide generation of an output by the model.

The term “generative artificial intelligence model” refers to a machine learning model that receives an input sequence and produces an output sequence, including at least a language model configured to generate natural-language text based on a prompt sentence.

The term “output format” refers to constraints or specifications regarding a structure, style, or length of text that is to be generated by the generative artificial intelligence model.

The term “recommendation content” refers to one or more suggestions or proposals generated by the generative artificial intelligence model, including at least nutritional supplementation advice or meal suggestions.

The term “advice text” refers to natural-language text generated by the generative artificial intelligence model that includes at least a recommendation relating to nutrition, diet, or physical activity for the user.

The term “advice history storage” refers to a data storage resource that holds one or more past advice texts in association with identifiers of users or sessions.

The term “emotional-index information” refers to data indicating an emotional condition of the user, the data being obtained from sensor readings, application inputs, or other sources, and expressing at least a level or type of emotion.

The term “self-report information” refers to information explicitly entered or confirmed by the user, such as text input or responses to questions, describing subjective states including mood or preferences.

The term “emotional state” refers to a condition of the user's emotion, such as stress level or mood, inferred by the processor based on the emotional-index information and the self-report information.

The term “meal menu” refers to a proposed combination of food and beverage items composing one or more meals, designed to satisfy nutritional or preference criteria.

The term “dialogue history data” refers to stored data representing previous exchanges between the user and the system, including prompts, responses, timestamps, and associated evaluation data.

The term “additional input text” refers to one or more text messages transmitted from the user to the server after initial advice has been provided, including follow-up questions, feedback, or constraints.

The term “response text” refers to natural-language text generated by the generative artificial intelligence model in reply to a prompt sentence that includes at least part of the dialogue history data.

The term “multi-turn, context-aware advice generation” refers to a process in which the system repeatedly exchanges messages with the user while maintaining and utilizing prior dialogue history and structured evaluation data to generate subsequent advice texts that reflect accumulated context.

In the following embodiments, the same reference to “server”, “terminal”, and “user” is used for clarity; however, these embodiments are merely illustrative and do not limit the scope of the claims.

The server includes at least one processor, a main memory, a non-volatile storage device, a network interface, and one or more hardware accelerators such as a graphics processing unit. The server executes one or more software components including an operating system, a web service framework, a database management system, a machine learning runtime, and an application program implementing the claimed functions. The terminal includes at least one processor, a memory, a display, a camera module as an imaging device, an acceleration sensor, a location information acquisition device, a wireless communication module, and an operating system that provides sensor and network APIs. The user operates the terminal via a graphical user interface of an application installed on the terminal.

The terminal acquires image data representing food and beverage ingested by the user.

The terminal uses its camera hardware controlled through an operating-system-specific framework, such as a camera control library, to convert optical information into digital pixels stored as an image file. The terminal can attach metadata such as capture time and location coordinates obtained from the location information acquisition device. The server receives the image file via a network connection and stores it in a storage device as part of a meal-image repository.

The server executes machine-learning-based image analysis by means of a trained image recognition model. The server uses a numeric computation framework, such as a deep learning runtime, to load a convolutional neural network configured for food recognition. In one embodiment, the server uses an architecture with multiple convolutional layers, batch-normalization layers, rectified-linear-unit activation functions, pooling layers, and fully connected layers followed by a softmax output layer. The server converts the image file into a tensor representation by resizing the image to a fixed resolution, normalizing pixel values, and arranging the values into a three-dimensional array. The server inputs the tensor into the neural network and computes feature maps through the convolutional layers. The server uses learned filter weights to extract local patterns corresponding to edges, textures, and shapes, and higher-level layers to extract semantic features representing specific dishes or ingredients.

The server determines a type of the food and beverage by selecting classes whose softmax probabilities exceed a threshold. The server determines an amount of each food or beverage item by applying additional estimation algorithms. In one embodiment, the server uses an auxiliary regression head attached to the neural network to output a continuous value representing portion size. In another embodiment, the server segments the image into regions using a segmentation network and estimates physical area ratios relative to known plate geometry to infer mass or volume. The server stores the recognized items and estimated amounts in structured records, such as database rows or key-value objects, associated with the user and the capture time.

The server calculates nutrient component amounts by referencing a nutrition information storage. The server uses a database management system to store a table in which standard food categories are associated with nutrient profiles, including energy, protein, fat, carbohydrates, and optionally additional nutrients. The server retrieves the appropriate row for each recognized food type and multiplies the standard nutrient values by the estimated amount to obtain actual nutrient component amounts for that meal. The server aggregates items within a meal and stores the aggregated nutrient component amounts as part of a dietary history data structure.

The terminal measures physical activity of the user using the acceleration sensor and the location information acquisition device. The terminal uses a sensor API to sample three-axis acceleration values at a configured frequency and a location API to acquire geographic coordinates over time. The terminal executes local filtering, such as low-pass filtering, to remove noise from acceleration signals and uses step-detection algorithms based on peak detection of acceleration magnitude. The terminal calculates movement distance and speed from successive location points using numerical approximations of geodesic distances. The terminal estimates energy expenditure based on body weight from user profile information and on movement distance and speed using empirically defined formulas. The terminal periodically formats this activity information into structured exercise records and transmits the exercise records to the server.

The server receives the exercise records and stores them in an exercise history storage.

The server can optionally execute additional classification of activity types by analyzing the temporal patterns of speed and acceleration. For example, the server can label intervals as walking, running, or resting based on speed thresholds and frequency-domain analysis of acceleration signals. The server maintains time-indexed records of movement distance, speed, activity type, and energy expenditure per interval, thereby forming physical activity history data.

The server evaluates nutritional status by combining dietary history data and physical activity history data over a predetermined period such as a day or a week. The server retrieves all meal records and exercise records for the target period from the corresponding databases. The server computes total intake energy as the sum of energy components from all meals and computes total energy expenditure as the sum of energy expenditure from all activity intervals. The server retrieves user profile information including age, sex, height, and weight and calculates basal metabolism using a predetermined formula. The server further determines a target energy balance based on user goals, such as weight maintenance or weight loss, and predetermined margins relative to basal metabolism.

The server compares intake energy and energy expenditure with the target energy balance and determines whether the user has an energy surplus or deficit. The server analyzes macro-nutrient balance by summing total protein, fat, and carbohydrate intakes and computing their ratios. The server applies rule-based criteria, such as thresholds for minimum daily protein or maximum saturated fat, to classify nutritional status into categories and to detect issues such as insufficient intake of specific nutrients. The server encodes the evaluation results into structured evaluation data, which may be represented as a record containing numeric fields and categorical fields for each type of detected condition.

The server constructs a natural-language text summarizing the structured evaluation data and the recent dietary and physical activity history. The server generates sentences describing key metrics, such as “Total intake energy today is 1,800 kcal, total energy expenditure is 2,000 kcal, and the net balance is a 200 kcal deficit.” The server includes user attributes such as age and target goals to contextualize the information. The server applies deterministic templates combined with data-driven selection of salient factors so that only relevant items are included, thus reducing the length of the text while preserving essential information.

The server constructs a prompt sentence for input to a generative AI model by appending instructions specifying the desired output format and recommendation content. For example, the server can generate a prompt sentence including the following text:

“The user is a 35-year-old male, 175 cm tall and weighing 70 kg, aiming to maintain current weight. Today's meals and exercise result in an energy intake of 1,800 kcal and an energy expenditure of 2,000 kcal, with slightly low protein intake and adequate carbohydrates and fats. Please provide practical nutrition supplementation advice for dinner, including three specific meal suggestions that increase protein intake while keeping total daily energy close to the maintenance level. Answer in English in about 200 words.”

The server can generate other prompt sentences adjusted to the user's emotional state and preferences. For example, the server can generate a prompt sentence such as:

“The user reports feeling tired and slightly stressed. Today the user skipped breakfast, ate a large high-fat lunch, and walked 3 km in the afternoon. Total intake energy is 1,500 kcal so far, with high fat and low fiber and vitamins. Please suggest two balanced dinner menus and one snack option that are easy to prepare, focus on vegetables and lean protein, and may help reduce fatigue, and briefly explain why.”

The server sends the prompt sentence to the generative AI model. In one embodiment, the server uses a transformer-based language model deployed on a separate inference server or cloud-based API. The generative AI model comprises multiple attention layers, feed-forward layers, and embedding layers trained on large-scale text corpora. During inference, the model tokenizes the prompt sentence into subword tokens, maps each token to an embedding vector, and processes the sequence through the attention layers to compute contextual representations. The model generates an output sequence by iteratively predicting the next token, conditioned on the prompt and previously generated tokens.

The server configures decoding parameters such as maximum token length, temperature, and nucleus sampling threshold, to control diversity and predictability of outputs. The server can implement a post-processing module that filters or normalizes the generated advice text to comply with length constraints and application policies. The server stores the advice text in advice history storage associated with a user identifier and a timestamp, thereby maintaining a log of past recommendations.

The server estimates an emotional state of the user using emotional-index information and self-report information. The terminal can collect emotional-index information through explicit user input, such as selecting an icon or completing a mood questionnaire, or through indirect sources, such as voice tone analysis or interaction patterns. The server maps these inputs to numeric or categorical values representing mood dimensions. The server applies thresholding and temporal smoothing to avoid reactivity to short-term fluctuations, and then encodes the resulting emotional state as part of the structured evaluation data or as a related structure.

The server incorporates the emotional state and the structured evaluation data into additional prompt sentences used to instruct the generative AI model to generate recommendations better adapted to the user's condition. By encoding emotional state and detailed nutritional context in a machine-optimized format, the server provides the generative AI model with a richer set of features than would be readily available from unstructured logs. This structuring reduces the number of model tokens needed to convey context, thereby lowering network bandwidth and inference time, and improves the consistency of generated advice.

The server manages dialogue history data to enable multi-turn, context-aware advice generation. The server records the sequence of prompt sentences and corresponding advice texts exchanged with the user. The server stores these records as structured entries containing at least timestamps, user messages, system-generated summarizations, and model outputs. When the user provides additional input text, such as follow-up questions, the server retrieves relevant portions of the dialogue history and the latest structured evaluation data. The server composes a new prompt sentence that includes a brief recap of earlier advice, the follow-up question, and any updated evaluation values. By summarizing past dialogue and limiting the context to the most relevant exchanges, the server reduces the length of the context delivered to the generative AI model, thereby improving computational efficiency and maintaining output relevance.

The server improves computer technology by implementing specific data structures and processing flows that optimize usage of hardware and network resources. For example, the server performs early aggregation of sensor data and image-derived nutrition data into compact structured evaluation data, rather than transmitting raw logs to external services. This aggregation reduces storage requirements and improves query times because the server can operate on aggregated records instead of large, unprocessed sequences. The server's use of bounding thresholds, early filtering, and structured representation of dialogue context reduces both the number of tokens processed by the generative AI model and the number of network requests, thereby reducing latency and communication load.

The server employs a training process for the neural network models that further enhances technical performance. During training, the server uses a dataset consisting of labeled food images and associated nutrition profiles. The server applies data augmentation techniques such as random cropping, rotation, scaling, and color jittering to increase robustness to variations in real-world usage conditions. The server defines a composite loss function combining classification loss and portion estimation loss, and uses gradient-based optimization, such as stochastic gradient descent or adaptive optimizers, to update model parameters. The server adjusts learning rates and regularization parameters to prevent overfitting and to improve generalization. This systematic training procedure yields a model that performs food recognition and portion estimation with higher accuracy on images captured under varied lighting and viewing angles, thereby directly improving the accuracy of downstream nutrient calculations.

The server can adopt alternative model architectures or processing variants while still remaining within the scope of the claims. In one embodiment, the server uses a residual network or a densely connected network for image analysis, where skip connections or dense connections facilitate gradient flow and deeper representations. In another embodiment, the server uses a vision transformer architecture that applies self-attention directly to image patches. In yet another embodiment, the server offloads part of the preprocessing, such as resizing and normalization, to the terminal to reduce computation on the server and decrease network payload size. In all cases, the server integrates the image-derived information with sensor-derived and profile-derived information in a unified evaluation pipeline.

The terminal can also implement alternative acquisition modes. In one variant, the terminal captures short video clips of meals instead of still images, and extracts representative frames for analysis. In another variant, the terminal collects higher-frequency acceleration data for short bursts to improve step detection and energy estimation accuracy. The terminal can switch sampling rates based on detected activity, thus reducing power consumption and unnecessary data generation when the user is inactive.

The system as described provides more than an abstract idea of collecting, analyzing, and displaying data. The server controls specific hardware components of the terminal, such as the imaging device, acceleration sensor, and location information acquisition device, under programmed logic to obtain raw physical measurements. The server transforms these raw signals into structured evaluation data using non-conventional, specific processing techniques involving neural network inference, composite loss training, rule-based nutritional evaluation, and optimized prompt construction. The server's management of dialogue context and prompt sentences is tied to the technical configuration of the generative AI model, leading to lower computational overhead and faster, more reliable advice generation. The integration of these components yields a technical improvement in health-related data processing systems, including improvements in processing speed, accuracy of nutritional estimation, data storage efficiency, and resource utilization during model inference.

The user ultimately receives advice through the terminal in a form that reflects both the structured evaluation data and the history of interactions. The user can view summaries of nutritional status, suggested meal menus, and explanations of the reasoning, while the underlying server continues to update models and databases in response to new image and sensor inputs. By virtue of the described architecture and algorithms, the system enables a more precise and efficient health management experience than would be achievable with conventional manual logging or simple rule-based recommendation systems, and concretely improves the functioning of the computers and AI components that implement the claimed system.

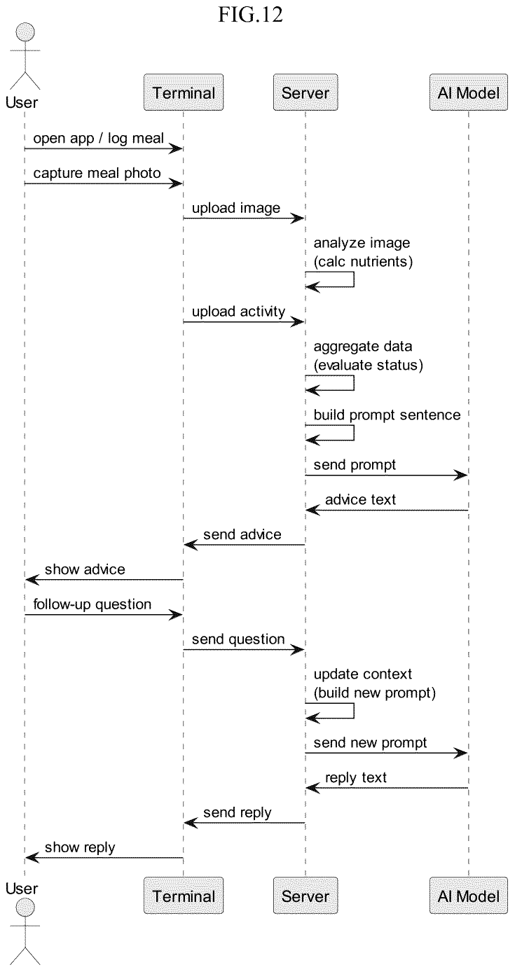

The following describes the processing flow using FIG. 12.

### Step 1:

The user operates the terminal to launch a health-management application and selects a meal logging function. The input is a user action on the graphical user interface, and the output is an internal application state indicating that the terminal is ready to capture a meal image. The terminal displays a camera preview screen and initializes connections to the imaging device, pre-allocating buffers to receive pixel data from the camera sensor.

### Step 2:

The terminal controls the imaging device to capture an image of food and beverage ingested by the user. The input is the optical scene of the meal and a capture command from the user, and the output is digital image data, such as a color image in a compressed file format. The terminal converts analog signals from the image sensor into digital pixel values, encodes the pixel array into an image file, and attaches metadata including a timestamp and position data obtained from the location information acquisition device.

### Step 3:

The terminal prepares and transmits an image upload request to the server. The input is the captured image file and metadata, and the output is a network message delivered to the server. The terminal packages the image data and associated metadata into a request structure, applies data compression if configured, and sends the request over a network protocol such as HTTPS to a designated server endpoint.

### Step 4:

The server receives the image upload request and stores the raw image data. The input is the network message containing the image and metadata, and the output is a stored image object and a corresponding record in a meal-image repository. The server parses the request, verifies user authentication, writes the image file to non-volatile storage, and registers an entry in a database associating the file path with the user identifier and timestamp.

### Step 5:

The server performs preprocessing on the stored image data for machine-learning-based analysis. The input is the stored image file, and the output is a normalized tensor suitable for neural network input. The server decodes the image into a pixel matrix, resizes the matrix to a predefined resolution, normalizes pixel intensities, and arranges the data into a multi-dimensional array with channels ordered according to the model specification.

### Step 6:

The server executes neural-network-based image analysis to identify a type and an amount of the food and beverage. The input is the normalized image tensor, and the output is a set of food category labels with associated probabilities and one or more portion-size values.

The server forwards the tensor through successive convolutional, activation, pooling, and fully connected layers, calculates feature maps and logits, applies a softmax function to obtain class probabilities, and optionally applies a regression head or a segmentation-based computation to estimate the quantity of each recognized item.

### Step 7:

The server maps recognized food categories and portion estimates to nutrition data. The input is the set of food labels and portion-size values, and the output is a list of nutrient component amounts for the meal. The server issues database queries to the nutrition information storage to retrieve nutrient profiles for each recognized category, multiplies per-unit nutrient values by the estimated portion sizes, and aggregates the results into a structured list of energy and nutrient quantities for each item.

### Step 8:

The server creates and stores a meal record in dietary history data. The input is the structured list of nutrient component amounts and associated item information, and the output is a new or updated meal record. The server constructs a data object containing the user identifier, timestamp, food types, portions, and aggregated nutrients, and then writes this object to a dietary history table using database insertion or update operations.

### Step 9:

The terminal measures physical activity using the acceleration sensor and the location information acquisition device. The input is raw sensor readings over time, and the output is processed activity data including movement distance, speed, and preliminary energy estimates. The terminal samples acceleration along multiple axes, filters noise from the time series, determines step counts, retrieves position coordinates at regular intervals, calculates distances between successive locations, computes average speeds, and applies energy-expenditure formulas based on user profile data.

### Step 10:

The terminal packages and transmits exercise records to the server. The input is the processed activity data for a period, and the output is a network message containing structured exercise records. The terminal segments the activity data into time intervals, labels each interval with estimated activity type, distance, speed, and calorie consumption, serializes these records into a structured format, and sends them to the server over the network.

### Step 11:

The server receives and stores the exercise records in physical activity history data. The input is the network message containing the activity records, and the output is one or more stored activity entries indexed by user and time. The server validates the content of the records, converts them into internal data structures, and executes database operations to insert or update corresponding entries in an exercise history storage.

### Step 12:

The server retrieves dietary and physical activity history data for a predetermined period. The input is a request trigger, which may be time-based or event-based, and the output is a set of meal records and activity records covering the target period. The server queries the dietary history data and physical activity history data using user-specific filters and time constraints, and loads the resulting records into application memory for further computation.

### Step 13:

The server computes aggregated intake energy and energy expenditure for the predetermined period. The input is the set of meal and activity records, and the output is total intake energy, total expenditure, and net balance values. The server sums energy values across meals to obtain intake, sums energy expenditure across activities to obtain output, and subtracts output from intake to compute the net energy balance for the time window.

### Step 14:

The server calculates basal metabolism and target energy balance using user profile information. The input is user profile data including age, sex, height, weight, and goals, and the output is a numeric basal metabolism value and a target energy range or value. The server applies a predefined metabolic formula to the profile data, adjusts for activity level and user goals, and stores the resulting baseline and target values for use in subsequent comparisons.

### Step 15:

The server evaluates nutritional status based on aggregated energy values, macro-nutrient totals, and target values. The input is the aggregated intake energy, energy expenditure, basal metabolism, target energy balance, and nutrient breakdowns, and the output is structured evaluation data representing the user's nutritional status. The server compares intake and expenditure against the target, determines surplus or deficit, checks macro-nutrient ratios against recommended ranges using rules, flags deficiencies or excesses, and compiles these results into a structured evaluation record containing numeric summaries and categorical indicators.

### Step 16:

The server estimates an emotional state of the user when emotional-index information and self-report information are available. The input is mood-related data obtained from the terminal and stored profile entries, and the output is a categorized emotional state value.

The server aggregates recent emotional inputs, smooths fluctuations using temporal averaging or thresholds, maps raw values into predefined categories such as “calm”, “stressed”, or “tired”, and stores the result as an emotional state attribute related to the user.

### Step 17:

The server generates a natural-language summary of the structured evaluation data and history data. The input is the structured evaluation data, the selected meal and activity records, user profile data, and optionally the emotional state, and the output is a compact natural-language text. The server selects key metrics, inserts numeric values into sentence templates, and constructs sentences that describe the user's energy balance, macro-nutrient balance, and notable patterns in food or activity, while ensuring that the total text length remains within a limit for efficient processing.

### Step 18:

The server constructs a prompt sentence for a generative AI model by combining the natural-language summary and explicit instructions. The input is the summary text and application-specific output requirements, and the output is a prompt sentence or multi-sentence prompt text. The server appends instructions describing the desired tone, level of detail, length, and type of recommendations. For example, the server can generate the following prompt sentence:

“The user is a 35-year-old male, 175 cm tall and weighing 70 kg, aiming to maintain current weight. Today's meals and exercise result in an energy intake of 1,800 kcal and an energy expenditure of 2,000 kcal, with slightly low protein intake and adequate carbohydrates and fats. Please provide practical nutrition supplementation advice for dinner, including three specific meal suggestions that increase protein intake while keeping total daily energy close to the maintenance level. Answer in English in about 200 words.”

### Step 19:

The server transmits the prompt sentence to the generative AI model and initiates text generation. The input is the prompt sentence, and the output is one or more sequences of generated tokens representing an advice text. The server sends the prompt over a communication interface to the model runtime, receives tokenized outputs produced by the model's internal transformer layers, and assembles the tokens into coherent sentences.

### Step 20:

The server post-processes and stores the advice text received from the generative AI model. The input is the raw generated text, and the output is a validated and formatted advice text stored in advice history storage. The server checks for compliance with length, language, and policy constraints, optionally trims or reformats sections into paragraphs or bullet points, associates the advice with user and time identifiers, and writes the resulting text into a dedicated advice history database.

### Step 21:

The server transmits the advice text to the terminal for presentation to the user. The input is the stored advice text and a delivery trigger, and the output is a network message carrying the advice to the terminal. The server selects an appropriate delivery mechanism, such as a push notification or a direct API response, encloses the advice text within a message structure, and sends it through the network interface.

### Step 22:

The terminal receives and displays the advice text to the user. The input is the network message containing the advice, and the output is a rendered user interface element presenting the advice on the display. The terminal parses the message, extracts the advice text, and displays it in a conversation view or dashboard, possibly highlighting key recommendations and providing interactive elements for user feedback.

### Step 23:

The user optionally provides additional input text in response to the advice. The input is the previously displayed advice and the user's intent to ask a follow-up question, and the output is a new user message submitted to the server. The user types or selects a query, such as “Can you recommend a vegetarian option with similar protein content?”, and the terminal encapsulates this input as a message linked to the current advice context.

### Step 24:

The server updates dialogue history data and generates a new prompt sentence for a follow-up interaction. The input is the additional user input text, the previous prompt and advice text, and the latest structured evaluation data, and the output is an updated dialogue history entry and a new prompt sentence. The server appends the new user message to the dialogue history, summarizes relevant past exchanges, and constructs a condensed prompt that includes the follow-up question and necessary context for the generative AI model.

### Step 25:

The server transmits the new prompt sentence to the generative AI model and obtains a follow-up response text. The input is the follow-up prompt sentence, and the output is a generated response text tailored to the follow-up query. The server repeats the transmission and generation steps, receives the newly generated tokens, assembles them into text, validates the content, and stores the result in the dialogue history and advice history stores.

### Step 26:

The terminal receives and displays the follow-up response text, enabling multi-turn, context-aware dialogue. The input is the network message containing the follow-up advice, and the output is an updated conversational screen showing the new response in sequence with prior messages. The terminal renders the conversation chronologically, allowing the user to review previous exchanges and to initiate additional steps of interaction as desired.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional nutrition management systems and wellness applications typically rely on manual entry of dietary records and physical activity logs by a user. Such approaches suffer from several technical limitations. First, manual input is error-prone and incomplete, leading to low-quality data that degrades the accuracy of downstream nutritional analysis algorithms. Second, even when image recognition and sensor-based activity tracking are used, processing is often implemented as isolated modules: image analysis, nutrition calculation, activity analysis, and advice generation are not tightly integrated at the system level. As a result, a processor cannot efficiently transform heterogeneous sensor data and image data into a coherent and machine-interpretable representation of a user's nutritional state.

In many existing systems, the generation of personalized guidance is implemented through fixed rule sets or static templates. Such implementations are not able to adaptively respond to complex combinations of food items, nutrient imbalances, and user-specific goals. When a generative AI model is used, the preparation of input data for the model (prompt engineering) is often ad hoc and manually configured, resulting in non-reproducible behavior, inconsistent quality of generated recommendations, and high computational overhead due to poorly structured prompts.

Furthermore, conventional systems do not exploit the full potential of combining structured nutritional computations with generative AI models. They typically pass only coarse summary data to a generative model, without encoding detailed structural relationships among ingredients, nutrient intake, energy expenditure, and user attributes. This limits the ability of the underlying computing system to produce context-appropriate and technically grounded recommendations, and leads to inefficient use of processing resources because the generative AI model must infer structure that could have been pre-computed by the processor.

There is therefore a need for a computer-implemented system that improves the way in which meal images, physical activity data, and user profile information are acquired, normalized, evaluated, and encoded as a structured prompt sentence for a generative AI model. In particular, there is a need to improve the functioning of the computer system itself by: (i) orchestrating interactions between external recognition resources, nutritional databases, and metabolic models; (ii) generating machine-interpretable nutrition state evaluation information in a consistent format; and (iii) automatically constructing prompt sentences that efficiently condition a generative AI model to output user-adapted guidance. Such improvements can reduce processing redundancy, increase consistency of outputs, and enable more precise control over the behavior of the generative AI model within a nutrition-management computing environment.

The specific processing by the specific processing unit 290 of the data processing device 12 in Example 2 is realized by the following means.

The present invention provides a server comprising a processor configured to acquire meal image data and physical activity data from a user terminal via a communication network and store the meal image data and the physical activity data in a storage device; to transmit the meal image data to an external processing resource for image recognition, identify food components based on an identification result from the external processing resource, and associate the food components with standardized food information; to acquire nutrition information corresponding to the standardized food information from a nutrition information storage unit and calculate, based on the nutrition information and estimated intake amounts of the food components, intake nutrient amounts for meals or for predetermined periods; to refer to metabolic equivalent information based on an exercise type, an exercise time, and body information included in the physical activity data, and calculate energy expenditure amounts for the predetermined periods by using the metabolic equivalent information; to compare the intake nutrient amounts and the energy expenditure amounts with predetermined reference information and user attribute information, and generate nutrition state evaluation information indicating an energy balance and surpluses or deficiencies of nutrients; to construct a prompt sentence for instructing a generative AI model to generate nutrition support proposals and eating habit improvement proposals, the prompt sentence being constructed based on the food components, the intake nutrient amounts, the energy expenditure amounts, the nutrition state evaluation information, and goal information of a user, and to transmit the prompt sentence to the generative AI model; and to format proposal information acquired from the generative AI model as guidance information in a natural language and notify the guidance information to the user terminal. This enables an improved computer-implemented nutrition management process in which heterogeneous input data are transformed into a structured, evaluation-based representation of a user's nutritional state, and in which a generative AI model is programmatically controlled through precisely constructed prompt sentences, thereby improving the consistency, efficiency, and technical quality of personalized nutrition guidance generated by the computing system.

The term “user terminal” refers to an information processing apparatus operated by a user, including but not limited to a portable terminal, a wearable terminal, or a stationary terminal, that is capable of capturing data related to meals and physical activity, executing an application program, and communicating with a server via a communication network.

The term “communication network” refers to a wired or wireless communication infrastructure, including local networks and wide-area networks, that enables data exchange between the user terminal, the server, and external processing resources.

The term “meal image data” refers to digital image information representing a meal consumed or to be consumed by the user, the digital image information being captured by an imaging device of the user terminal and including at least pixel values and associated metadata.

The term “physical activity data” refers to digital information representing physical activity performed by the user, the digital information including at least an exercise type, an exercise duration, and optionally body-related sensor data and location-related data.

The term “storage device” refers to a hardware resource or a combination of hardware resources, such as a semiconductor memory, a magnetic storage medium, or an optical storage medium, configured to store meal image data, physical activity data, and processing results in a non-transitory manner.

The term “external processing resource” refers to an information processing resource that is logically or physically separate from the server, including a remote computing resource or an externally provided function, that is configured to perform image recognition processing on meal image data and output identification results.

The term “image recognition” refers to computational processing applied to image data to detect and classify visual elements, including at least food items or dishes, and to output corresponding labels or identifiers with associated reliability information.

The term “food component” refers to a constituent element of a meal, including a food item, an ingredient, or a dish component, that can be identified from meal image data and associated with standardized food information.

The term “standardized food information” refers to normalized data representing food components, including a food identifier, a food category, and reference nutrient content per unit, stored in a predetermined format and used as a common reference across processing modules.

The term “nutrition information” refers to structured data describing nutrient content of a food component per unit quantity, including energy information and macronutrient and micronutrient information, and stored in a nutrition information storage unit.

The term “nutrition information storage unit” refers to a logical storage region within the storage device or an external storage resource that retains standardized food information and associated nutrition information for use by the processor.

The term “estimated intake amount” refers to a quantity value of a food component inferred or determined for a user's meal, obtained from image recognition results, predefined serving rules, or user input, and used for calculating intake nutrient amounts.

The term “intake nutrient amount” refers to a quantity of a nutrient ingested by the user for a meal or for a predetermined period, calculated based on nutrition information and the estimated intake amounts of corresponding food components.

The term “exercise type” refers to a classification of a physical activity performed by the user, including but not limited to walking, running, cycling, or strength training, which is used as a parameter for energy expenditure calculation.

The term “exercise time” refers to a time duration or time interval during which a physical activity is performed by the user, the time duration or time interval being included in or derived from the physical activity data.

The term “body information” refers to user-specific physiological or anthropometric data, including at least body weight and optionally age, height, or biological sex, which are used together with physical activity data for computing energy expenditure.

The term “metabolic equivalent information” refers to reference data representing a unitless metabolic intensity index associated with an exercise type and an activity intensity, used to convert physical activity into energy expenditure.

The term “energy expenditure amount” refers to a calculated quantity of energy consumed by the user due to physical activity over a predetermined period, the quantity being computed using metabolic equivalent information, body information, and exercise time.

The term “reference information” refers to baseline or target values used for comparison with calculated intake nutrient amounts and energy expenditure amounts, including recommended nutrient intake ranges and recommended energy intake ranges.

The term “user attribute information” refers to stored information characterizing the user, including at least demographic information, physiological information, lifestyle information, and dietary preference information, used to customize evaluations and guidance.

The term “nutrition state evaluation information” refers to structured data generated by the processor that describes an evaluation of a user's nutritional condition, including an energy balance and surpluses or deficiencies of individual nutrients relative to reference information.

The term “goal information” refers to stored information representing one or more objectives specified for the user, including at least body weight goals, body composition goals, or health maintenance goals, which influence evaluation and guidance generation.

The term “prompt sentence” refers to a text sequence or a structured natural-language description constructed by the processor, the text sequence encoding food components, intake nutrient amounts, energy expenditure amounts, nutrition state evaluation information, and goal information, and serving as input to a generative AI model.

The term “generative AI model” refers to a machine-implemented probabilistic model, including a neural network-based language model, trained to generate natural-language text in response to an input prompt sentence.

The term “nutrition support proposal” refers to generated content that recommends adjustments or actions related to nutrient intake, including suggestions to increase or decrease particular nutrient categories or food categories.

The term “eating habit improvement proposal” refers to generated content that recommends modifications to recurring dietary behaviors of the user, including meal timing, food selection patterns, and portion control strategies.

The term “proposal information” refers to data output by the generative AI model in response to a prompt sentence, the data including nutrition support proposals and eating habit improvement proposals in a machine-processable form.

The term “guidance information” refers to human-readable natural-language information derived from proposal information, arranged in a format suitable for display to the user on the user terminal.

The term “psychological information” refers to data representing a user's mental or emotional state or tendencies, inferred from user interaction patterns, self-reports, or behavioral indicators, and used to adjust the style and content of guidance.

The term “behavior history information” refers to data representing a sequence of past actions by the user, including past meals, past physical activities, and past responses to guidance, stored and used for personalization of subsequent guidance.

The term “expression style” refers to linguistic characteristics of guidance information, including tone, politeness level, directness, and use of technical terminology, which are controlled by the processor.

The term “level of detail” refers to a granularity of explanation provided in guidance information, including the amount of technical explanation and the number of concrete recommendations presented to the user.

The term “priority of recommended content” refers to an ordering or weighting of individual recommendations within proposal information, used to emphasize or de-emphasize particular suggestions in the guidance information.

The term “dialogue history information” refers to stored records of previous exchanges between the user terminal and the system, including prior user queries and prior system responses, used by the processor to maintain conversational context.

The term “response format specification information” refers to control data embedded in a prompt sentence that instructs the generative AI model regarding a desired output structure, including but not limited to paragraph structure, list formats, and response length.

The term “dialogue management processing” refers to processing executed by the processor to manage stateful interactions with the user, including maintaining dialogue context, selecting appropriate prompt sentences, routing responses, and updating dialogue history information.

In one embodiment, a server, a user terminal, and one or more external processing resources cooperate to implement the invention. The server includes at least one processor, a main memory, a non-transitory storage device, and a network interface. The terminal includes an image capture device, a user interface, a local storage device, and a communication module. The external processing resources include an image recognition system and, in some embodiments, an external generative AI model endpoint. The server executes a nutrition-management program stored in the storage device; the terminal executes a client-side application program.

The terminal uses camera hardware and an operating system camera API, such as a generic camera framework comparable to AVFoundation or CameraX, to obtain meal image data. The terminal stores the captured image in local storage and then transmits the image data, together with metadata such as timestamp and meal category, to the server via a communication network using a secure protocol such as HTTPS. The server receives this data through a web server component, such as a generic HTTP server, and forwards it to an application framework implemented for example in a runtime environment such as a generic scripting runtime or a managed language runtime.

The server stores the raw meal image data in a storage device such as a disk array or a network-attached storage system, and stores metadata in a database system such as a relational database. The database holds structured tables for users, meals, ingredients, standardized food information, nutrition information, physical activity records, metabolic equivalent values, evaluation results, and advice records. Each table has defined fields; for example, a meal record includes a meal identifier, a user identifier, a timestamp, and a pointer to the image file.

The server transmits the meal image data or a reference thereto to an external image recognition resource. This external resource can be implemented as a general-purpose image recognition API deployed on remote computing infrastructure. The external resource executes a neural-network-based image recognition algorithm, for example a convolutional neural network (CNN) with multiple convolutional layers, pooling layers, and fully connected layers trained on labeled food image datasets. The external resource returns a structured identification result that includes food labels, confidence scores, and optionally bounding box coordinates.

The server parses the identification result and maps the labels to standardized food information. The server uses a mapping table stored in the database, where each label from the image recognition resource corresponds to a standardized food identifier. The standardized food information includes category fields, a normalization key, and a link to nutrition information. This mapping reduces variability in label expressions and improves the consistency of downstream calculations, which directly enhances the technical reliability of the nutrition computation process.

The server retrieves nutrition information corresponding to the standardized food identifiers from a nutrition information storage unit. The nutrition information contains per-unit nutrient values for energy, macronutrients, and micronutrients. The server calculates an estimated intake amount for each food component. In one embodiment, the server uses bounding box size and relative area within the image, combined with heuristic rules and user profile information (for example, typical portion size based on user body weight), to estimate a portion factor. The server then multiplies the per-unit nutrient values by the portion factor to obtain per-ingredient nutrient quantities, and sums them to generate intake nutrient amounts at meal level and at day level. These operations are implemented as numerical computations in the processor using arithmetic operations and, optionally, numerical libraries.

The terminal or a wearable device supplies physical activity data to the server. The terminal collects exercise type, start time, end time, and optionally heart rate information from sensor APIs such as motion sensors and location services. The terminal transmits the physical activity data to the server via the communication network. The server stores the raw physical activity data in the database and then calculates energy expenditure amounts.

The server references a metabolic equivalent table that maps exercise types and intensity levels to metabolic equivalent values. The server retrieves body information, such as user weight, from a user profile table. The server computes energy expenditure for each activity by multiplying the metabolic equivalent, the body weight, and the exercise time in hours, and then aggregates the results for predetermined periods such as one day.

The server compares the calculated intake nutrient amounts and energy expenditure amounts with reference information and user attribute information. The reference information includes recommended nutrient intake ranges and energy intake ranges, stored in a guideline table. The user attribute information includes age group, sex, health goal, and dietary preferences. The server determines, for each nutrient, whether the intake is within, below, or above the recommended range; similarly, the server determines whether the net energy balance is positive, negative, or near zero. The server encodes the evaluation result as nutrition state evaluation information, which contains structured fields indicating, for example, “calorie surplus: yes/no,” “protein deficiency: degree,” or “fiber deficiency: degree.”

The server uses this structured evaluation information to construct a prompt sentence for a generative AI model. The server concatenates fields such as identified food components, intake nutrient amounts, energy expenditure amounts, nutrition state evaluation information, and user goals into a text representation in natural language. The prompt sentence may include explicit labels for each section to provide a machine-readable structure to the generative AI model. The prompt sentence controls which information is highlighted for the generative AI model and constrains the output style and level of detail. In one embodiment, the generative AI model is a large-scale language model implemented as a transformer neural network with an encoder-decoder or decoder-only architecture. The model uses multi-head self-attention layers, feed-forward layers, positional encodings, and layer normalization. The model is trained on a large corpus of text including general language data and, optionally, specialized nutrition and health-related documents. During training, the model minimizes a cross-entropy loss function between predicted tokens and ground-truth tokens, and updates weights using a gradient-based optimization algorithm such as stochastic gradient descent or a variant such as Adam. Data augmentation methods, including random masking and shuffling of segments, can be used to improve robustness. By describing the underlying model in terms of layers, loss functions, and optimization procedures, the implementation clarifies that the generative AI model is a specific technical construct rather than an abstract decision entity.

The server calls the generative AI model through an inference API. The server transmits the constructed prompt sentence and parameters such as maximum output length, temperature, and response format specification. The generative AI model processes the prompt sentence with its attention layers, generating token probabilities conditioned on the structured nutritional context. Because the prompt sentence encodes pre-computed numerical results, the generative AI model is relieved from inferring quantities from raw images or raw sensor data, which reduces computational burden and improves the accuracy and stability of its responses. This constitutes a technical improvement in the functioning of the combined system: the workload is divided between deterministic numeric computation on the server and probabilistic natural-language generation in the generative AI model.

The server receives the output tokens from the generative AI model and reconstructs the proposal information as text. The server optionally performs post-processing to normalize units, detect and remove inconsistent or out-of-scope statements using rule-based filters, and segment the advice into bullet points or sections. The server stores the finalized guidance information in the database and transmits a notification to the terminal. The terminal displays the guidance information through the user interface, allowing the user to review detailed nutritional recommendations and explanations generated on the basis of computed evaluation results and goals.

In another embodiment, the server adjusts the expression style, level of detail, and priority of recommended content included in the prompt sentence. The server maintains psychological information and behavior history information for the user in the database. The psychological information may include sensitivity to strict wording or preference for encouraging language; the behavior history information may include which previous recommendations were followed and which were ignored. The server encodes these parameters into the prompt sentence, for example by adding instructions such as “use concise, encouraging language” or “focus on two priority recommendations related to fiber and total energy.” By dynamically modifying the prompt sentence structure and content based on stored user-specific parameters, the server controls the generative AI model in a deterministic and reproducible manner, which enhances the technical consistency of generated guidance and reduces the need for manual tuning.

In yet another embodiment, the server includes dialogue history information and response format specification information in the prompt sentence. The server maintains a dialogue log in the database, including prior user questions and system responses. When a new interaction is initiated, the server selects relevant portions of the dialogue history and embeds them into the prompt sentence as context, along with instructions specifying that the response should be in a conversational format, such as a short question-and-answer style or a numbered list with explanations. The generative AI model then produces a conversational response that takes into account the context and the format constraints. The server uses a dialogue management module to route the conversational responses to the terminal and to update the dialogue history table. This system-level design improves the technical behavior of the dialogue mechanism, including state tracking and consistency of responses across multiple turns.

A concrete example of a prompt sentence constructed by the server is as follows:

“The server has analyzed the following data for one user.

Meals today include: salad with lettuce, tomato, and grilled chicken.

Estimated total intake: 1,850 kcal, 65 g protein, 70 g fat, 210 g carbohydrates, low dietary fiber.

Exercise today: 45 minutes of jogging, calculated energy expenditure 350 kcal.

User goal: gradual weight loss and improved cardiovascular fitness.

Based on this information, evaluate the user's nutrition status and generate clear, practical advice on how to adjust tonight's dinner and tomorrow's meals. Suggest specific foods to increase or decrease, and include brief suggestions for exercise. Respond in concise

English, suitable for display in a mobile app.”

In another example, the server constructs a prompt sentence that emphasizes adaptation of expression style:

“The system has determined that the user prefers short, encouraging messages and has difficulty following more than two recommendations at a time.

Current evaluation: slight calorie surplus, low dietary fiber, adequate protein intake.

User goal: reduce body weight by 0.5 kg per week.

Provide at most two key recommendations in positive and encouraging language. Focus on increasing fiber intake and slightly reducing overall calories, without reducing protein. Avoid technical jargon and keep the advice brief.”

By constructing prompt sentences in this way, the server imposes a structured and technically meaningful conditioning on the generative AI model. The prompt sentences encode numeric and categorical results produced by deterministic algorithms, and define explicit output constraints. This cooperation between numeric computation and generative modeling yields technical effects such as reduced latency in inference, because the generative AI model no longer needs to parse raw sensor data; improved accuracy of guidance, because numeric evaluations are performed by dedicated algorithms using verified databases; and reduced network overhead, because the server transmits compact structured text instead of large raw data to the generative AI model.

From a system architecture perspective, the server can be implemented as multiple cooperating modules: an ingestion module for receiving meal image data and physical activity data; an image analysis integration module for interfacing with external image recognition resources; a nutrition calculation module for computing intake nutrient amounts; an activity analysis module for computing energy expenditure amounts; an evaluation module for generating nutrition state evaluation information; a prompt generation module for constructing prompt sentences; an AI integration module for interacting with the generative AI model; and a delivery module for formatting and transmitting guidance information to the terminal. Each module operates on defined data structures such as normalized ingredient lists, nutrient vectors, activity records, and evaluation records, which are stored in and retrieved from database tables. This modular, data-driven design improves maintainability, facilitates scaling, and permits optimization of computation and communication paths.

The technical effect of the invention arises from the specific cooperation of these modules and data structures. The use of standardized food information and nutrition information tables reduces data redundancy and permits efficient indexing and caching, which accelerates nutrient computations. The separation of image recognition into an external processing resource and the use of a pre-mapped label dictionary reduce misclassification errors and enable the server to reuse recognition results across different analysis tasks. The generation of nutrition state evaluation information as a structured, machine-interpretable object allows the prompt generation module to create reproducible and optimally informative prompt sentences for the generative AI model. As a result, the generative AI model can generate high-quality, context-aware advice with fewer tokens and lower randomness, thereby enhancing both throughput and reliability.

In addition, the server can employ non-conventional pre- and post-processing rules that differ from simple human reasoning patterns. For example, the server can apply weighting schemes to prioritize certain nutrients based on recent patterns of excess or deficiency, can cluster meals using vector representations of ingredients to detect repetitive dietary patterns, and can alter the prompt structure accordingly. These machine-oriented rules and data transformations are specifically designed to improve model conditioning and not to mimic manual counseling procedures. Therefore, the system does not merely automate human nutrition counseling but instead provides a new computational method that improves the functioning of the underlying computer system in terms of processing speed, accuracy, and resource utilization.

Alternative embodiments are possible. The generative AI model may be hosted on the same physical device as the server, using dedicated accelerator hardware such as general-purpose graphics processors or tensor processors. In such an embodiment, the server can share memory buffers directly with the model, further reducing communication overhead. The image recognition resource may also be implemented as a local module running on the server using a pre-trained CNN model. The nutrition information storage unit may be distributed over multiple physical storage devices and accessed through a caching layer to reduce database query latency. The evaluation algorithms may be extended to handle additional biomarkers or real-time sensor data such as continuous glucose monitoring signals.

In another variant, the terminal can perform preliminary image down-sampling and compression to reduce uplink bandwidth, while preserving sufficient resolution for the external image recognition resource. The terminal can also perform local caching of guidance information and history to enable offline browsing and reduce redundant data retrieval from the server. These configurations demonstrate that the invention can be adapted to different hardware and deployment environments while preserving the core technical features: structured numeric evaluation of nutritional and activity data, and controlled use of a generative AI model via programmatically constructed prompt sentences.

Through these implementations, the server, the terminal, and the external processing resources cooperate to provide a computer-implemented nutrition management system that goes beyond abstract data processing or simple automation of human tasks. The system improves internal data representation, processing efficiency, and communication patterns in a way that directly enhances the technical performance of the computing environment.

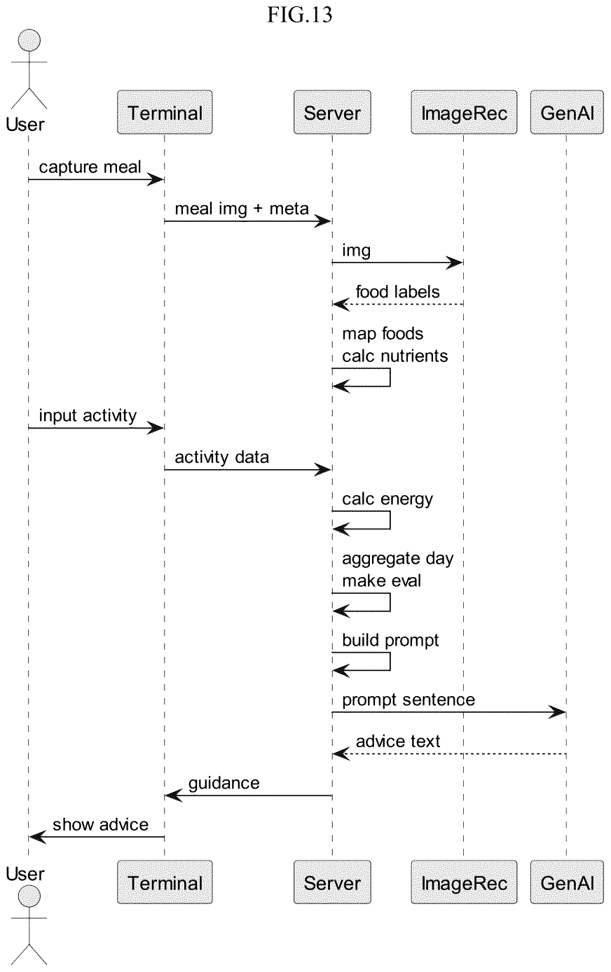

The following describes the processing flow using FIG. 13.

### Step 1:

The user uses the terminal to capture a meal image and provide basic meal metadata.

The terminal receives, as input, raw pixel data from a camera sensor and user selections such as meal type and time.

The terminal uses an operating system camera API to convert the sensor data into a compressed image file (for example, JPEG) and writes the file into local storage, generating an output consisting of an image file path, associated metadata (timestamp, meal type), and a user identifier.

The terminal then prepares an HTTPS request body that embeds the image file binary and the metadata as multipart/form-data, which becomes the output of this step for transmission to the server.

### Step 2:

The terminal transmits the meal image data and metadata to the server.

The terminal takes, as input, the locally stored image file path, the meal metadata, and an authentication token previously issued by the server.

The terminal reads the image file into memory, attaches the metadata and token to a POST request, and sends the request over a communication network using HTTPS.

The output of this step is an authenticated HTTP request containing the image data and metadata, which is delivered to the server's network interface.

### Step 3:

The server receives, validates, and stores the meal image data and metadata.

The server takes, as input, the HTTP request from the terminal containing the image binary, metadata, and authentication token.

The server uses an authentication module to validate the token, checks the image format and file size, and then writes the raw image to a storage device, such as a file system or object storage, while inserting a meal record into a database with fields including a meal ID, user ID, image path, timestamp, and meal type.

The output of this step is a persistent meal record and a stored image file associated with that meal record.

### Step 4:

The server sends the stored meal image to an external image recognition resource and receives identification results.

The server takes, as input, the meal record and the corresponding image path in storage.

The server reads the image or generates an access URL, constructs an API request for an external image recognition resource, and sends the request over the communication network.

The external resource applies an image recognition algorithm, such as a convolutional neural network, and returns a structured response containing food labels, confidence scores, and optional bounding boxes.

The output of this step is a machine-readable identification result received by the server as a data structure, for example a list of label-confidence pairs.

### Step 5:

The server maps the identification results to standardized food information.

The server takes, as input, the image recognition labels and confidence scores.

The server filters out labels with confidence scores below a threshold and then queries a mapping table in the database that relates external labels to standardized food identifiers.

The server performs table lookups and joins to associate each filtered label with a standardized food ID, food category, and reference portion information.

The output of this step is a standardized ingredient list that includes food IDs, normalized names, categories, and confidence values, stored in an ingredient table linked to the meal ID.

### Step 6:

The server retrieves nutrition information and calculates intake nutrient amounts.

The server takes, as input, the standardized ingredient list for the meal.

The server queries a nutrition information table that stores per-unit nutrient values (for example, per 100 g) for each standardized food ID. The server then estimates a portion size for each ingredient, based on reference portion information, bounding box area ratios, or user-provided portion hints if available. The server multiplies the per-unit nutrient values by the estimated portion factors to compute nutrient values per ingredient, and sums across all ingredients to obtain total energy and nutrient amounts for the meal.

The output of this step is a nutrient profile for the meal, stored in the database as intake nutrient amounts associated with the meal ID and user ID.

### Step 7:

The user provides physical activity information through the terminal or a wearable-linked application.

The terminal takes, as input, user actions such as selecting an exercise type and specifying start and end times, or synchronized activity records from a wearable device interface.

The terminal aggregates the exercise information into a structured physical activity record containing exercise type, duration, intensity level, and optionally heart rate and location samples.

The output of this step is a physical activity data object, which the terminal prepares for transfer to the server in an HTTPS request.

### Step 8:

The terminal transmits the physical activity data to the server.

The terminal takes, as input, the structured physical activity record and the user's authentication token.

The terminal serializes the activity record into a JSON or similar payload, attaches the authentication token, and sends a POST request to an activity endpoint on the server over HTTPS.

The output of this step is an authenticated HTTP request that delivers the physical activity data to the server's network interface.

### Step 9:

The server receives, stores, and normalizes the physical activity data.

The server takes, as input, the HTTP request containing the activity data and authentication token.

The server validates the token, parses the payload, and writes the physical activity entry into an activity table in the database, recording user ID, exercise type, start time, end time, intensity, and any sensor metrics such as heart rate. The server may normalize exercise type to a standardized exercise code using a lookup table.

The output of this step is a normalized physical activity record stored in the database and linked to the user.

### Step 10:

The server calculates energy expenditure amounts from the physical activity data.

The server takes, as input, the normalized physical activity record and body information for the user (such as weight and age) from the user profile table.

The server queries a metabolic equivalent table to derive a metabolic equivalent value for the combination of exercise type and intensity, converts the activity duration from timestamps into hours, and computes energy expenditure using a formula that multiplies the metabolic equivalent by user weight and duration. The server stores the resulting energy expenditure amount back into the activity record or into a separate energy expenditure table.

The output of this step is a computed energy expenditure value per activity session and, by aggregation, per predetermined period such as a day.

### Step 11:

The server aggregates intake nutrient amounts and energy expenditure amounts for a predetermined period.

The server takes, as input, all meal nutrient profiles and energy expenditure values for the user within a selected time window, such as one calendar day.

The server runs aggregation queries over the meal and activity tables, summing energy and individual nutrient values for intake, and summing calories for expenditure. The server also calculates a net energy balance as total intake minus total expenditure. The output of this step is an aggregated daily (or period-based) nutrition and energy summary stored as a record in an evaluation table.

### Step 12:

The server generates nutrition state evaluation information.

The server takes, as input, the aggregated nutrition and energy summary, reference information (such as recommended intake ranges), and user attribute information (such as goals and dietary restrictions).

The server compares each nutrient's intake against corresponding reference ranges and classifies each as deficient, adequate, or excessive. The server similarly categorizes the energy balance as surplus, deficit, or within target. These comparisons are implemented as conditional evaluations and threshold checks. The server encodes the results into a structured evaluation object containing fields for energy balance and per-nutrient status. The output of this step is nutrition state evaluation information stored in the database and linked to the user and period.

### Step 13:

The server constructs a prompt sentence for a generative AI model.

The server takes, as input, the standardized ingredient list, intake nutrient amounts, energy expenditure amounts, nutrition state evaluation information, user goal information, and optionally psychological and behavior history information.

The server formats these inputs into a structured natural-language text by concatenating descriptive phrases and numeric values into labeled sections, and by embedding instructions that specify tone, level of detail, and output format. The server may include constraints such as maximum number of recommendations and target language.

The output of this step is a prompt sentence that encodes the computed numerical and categorical data in a machine-interpretable natural-language form, ready to be sent to the generative AI model.

### Step 14:

The server transmits the prompt sentence to the generative AI model and receives proposal information.

The server takes, as input, the constructed prompt sentence and model parameters such as maximum token count and randomness control values.

The server creates an API request to a generative AI model endpoint, supplying the prompt sentence and parameters, and sends this request over the communication network. The generative AI model executes its internal transformer-based inference to generate a sequence of tokens that form natural-language recommendations. The server receives the token sequence and reconstructs it into text.

The output of this step is proposal information consisting of nutrition support proposals and eating habit improvement proposals in textual form.

### Step 15:

The server formats the proposal information into guidance information suitable for the user terminal.

The server takes, as input, the raw text proposals returned by the generative AI model and formatting rules defined in the application.

The server parses the text to segment recommendations, applies rule-based filters to remove disallowed or inconsistent statements, and adds structural markers such as headings or bullet indicators. The server may also annotate the guidance with references to specific meals or days. The server stores the resulting guidance text in the database as a guidance record linked to the user and relevant period.

The output of this step is finalized guidance information ready for distribution to the terminal.

### Step 16:

The server notifies the terminal and provides the guidance information.

The server takes, as input, the guidance record and the user's device registration information for push notifications.

The server sends a short notification message via a push notification service, including an identifier of the guidance record. When the terminal later requests the full guidance, the server retrieves the guidance text from the database and returns it in an HTTP response.

The output of this step is a delivered response containing the full guidance information, which the terminal can display to the user.

### Step 17:

The terminal displays the guidance information and facilitates user interaction.

The terminal takes, as input, the HTTP response containing the guidance text.

The terminal renders the text in the user interface, arranging recommendations as readable sections or lists. The terminal may record user actions such as marking recommendations as completed or requesting clarifications, and may subsequently send this feedback to the server to be used as behavior history information in future processing.

The output of this step is an updated user interface state showing the guidance and, optionally, interaction logs that can be reused by the server in later evaluation and prompt sentence generation.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional health management systems that log meals and exercise typically treat sensing, analysis, and feedback as loosely coupled and manually configured components. In many cases, such systems merely store meal photos and step counts and then display static dashboards. As a result, a processor in those systems must execute multiple disjoint workflows: image analysis, nutrient lookup, exercise energy estimation, and message generation, each often implemented as separate services or manually triggered routines. This fragmentation leads to high latency, duplicated data processing, inconsistent context between components, and difficulty in scaling computation when user data volume increases.

Furthermore, existing approaches that attempt to incorporate machine learning models for nutritional advice often rely on fixed, preauthored templates or rigid rule engines. In such architectures, the processor does not construct machine-interpretable, context-rich prompt sentences that unify multi-modal inputs (image data, sensor data, text data) and historical health information. Instead, a separate client application or human operator manually summarizes data and calls a generative AI model. This separation causes inefficiencies in network usage, repeated serialization of similar input data, and loss of fine-grained context such as temporal patterns of deficiency or emotional trends. The generative AI model is therefore not fully leveraged as an adaptive component within the computing pipeline, and the underlying computing resources are not efficiently utilized.

In addition, conventional systems treat emotion estimation, nutritional status evaluation, and dialogue management as separate problem domains. Emotion information, if used at all, is typically processed by an independent module that is not tightly integrated with the construction of the generative AI prompt sentence or with downstream adjustment of recommendations. As a consequence, the processor cannot systematically adjust the structure and content of prompt sentences and resulting dialog according to both nutritional status and emotional state. This leads to repeated or redundant calls to external services, unnecessary data transformations, and increased processing overhead.

There is therefore a need for a technical architecture in which a processor integrates image-based meal recognition, sensor-based exercise analysis, user attribute management, temporal health aggregation, emotion estimation, and generative AI interaction within a single coordinated data flow. The processor should itself construct and update prompt sentences for a generative AI model from normalized, structured evaluation data, and should post-process the generated advice according to algorithmic rules or learned models. By doing so, the computing system can reduce duplicated computation, better reuse intermediate representations, dynamically adapt prompt construction and dialog flow to current states, and thereby improve overall efficiency, scalability, and consistency of the health management computation.

The specific processing by the specific processing unit 290 of the data processing device 12 in Application Example 2 is realized by the following means.

The present invention provides a server comprising a processor configured to acquire, from a terminal, meal information of a user as image information, perform object recognition processing and feature extraction processing on the image information to specify ingredient information, estimate intake nutrient information by referring to a nutrient information storage unit, acquire exercise information from motion sensors and position information units, compute activity intensity information and energy expenditure information, integrate the intake nutrient information and the energy expenditure information with user attribute information and health-related information over a predetermined period to generate nutritional status evaluation information, automatically construct a context-rich prompt sentence including evaluation summary information and output condition information, supply the prompt sentence as input to a generative information processing model, receive advice information output from the generative information processing model, and perform content correction processing and expression adjustment processing on the advice information to generate user-oriented nutrient supplementation advice information, wherein the processor is further configured to acquire emotion information of the user, apply an emotion estimation model to calculate emotion state information, modify the configuration of the prompt sentence based on the emotion state information, and execute bidirectional dialog processing with the user by repeatedly generating updated prompt sentences that incorporate additional user inputs and storing user responses as components of subsequent prompt sentences. This enables the server to centralize and orchestrate multi-modal data processing and generative model interaction within a unified computational pipeline, reduce redundant preprocessing and network communication, dynamically adapt prompt construction and recommendation content to both nutritional status and emotional state, and thereby improve the efficiency, responsiveness, and contextual consistency of computer-implemented health management processing

The term “meal information” refers to information representing food or beverages consumed or to be consumed by a user, including but not limited to images, textual descriptions, and associated metadata such as time, location, and context of intake.

The term “image information” refers to digital data representing a visual scene, including but not limited to still images encoded in a raster format and associated metadata such as resolution, color space, and capture time.

The term “terminal” refers to an information processing apparatus operated by a user, including but not limited to a mobile communication device, a portable computing device, or a wearable computing device capable of capturing sensor data and communicating with a server.

The term “object recognition processing” refers to processing in which an algorithm analyzes image information to detect and classify visual objects, such as food items, and outputs identifiers or labels representing the detected objects.

The term “feature extraction processing” refers to processing in which an algorithm calculates numerical or symbolic representations from input data, such as image information, for use in object recognition, classification, or further analysis.

The term “ingredient information” refers to data representing constituent items of a meal, including but not limited to types of food, categories of food, and estimated quantities or portions associated with each item.

The term “nutrient information storage unit” refers to a data storage component, such as a database or memory structure, that stores nutrient data associated with food or ingredient identifiers, including values for energy, macronutrients, and micronutrients.

The term “intake nutrient information” refers to estimated or calculated data representing nutrients ingested by a user during one or more meals, including quantities of energy, macronutrients, micronutrients, and other nutritional components.

The term “exercise information” refers to data representing physical activity of a user, including but not limited to activity type, duration, distance, speed, steps, and physiological indicators captured by sensors.

The term “motion sensor” refers to a sensing component that detects movement or acceleration of a device or a body, including but not limited to an accelerometer, a gyroscope, or a combined inertial sensor.

The term “position information acquisition unit” refers to a component or subsystem that acquires geographical position data, including but not limited to a satellite positioning module, a network-based positioning module, or a combination thereof.

The term “activity intensity information” refers to information indicative of the intensity level of physical activity, derived from exercise information and user attributes, and expressible, for example, in terms of metabolic equivalents or similar indicators.

The term “energy expenditure information” refers to estimated or calculated data representing the amount of energy expended by a user as a result of physical activity, typically expressed as a quantity of calories or equivalent units.

The term “user attribute information” refers to information that characterizes a user and affects health-related computation, including but not limited to age, sex, height, weight, lifestyle, and preference data.

The term “health-related information” refers to information related to the health condition or health behavior of a user over time, including but not limited to meal history, exercise history, sleep data, biometric data, and self-reported conditions.

The term “nutritional status evaluation information” refers to data representing an assessment of a user's nutritional state, derived from intake nutrient information, energy expenditure information, user attribute information, and health-related information.

The term “nutrient sufficiency information” refers to information indicating whether quantities of nutrients ingested by a user meet, exceed, or fall short of predetermined reference values over a specified period.

The term “energy balance information” refers to information representing a relation between energy intake and energy expenditure of a user over a predetermined period, including but not limited to surplus or deficit values.

The term “reference information” refers to baseline or recommended values used in evaluating nutritional or energy status, including but not limited to dietary reference intakes and target activity levels.

The term “evaluation summary information” refers to condensed information derived from detailed evaluation data, including at least deficiency nutrient information and excess nutrient information, suitable for input to a generative model.

The term “deficiency nutrient information” refers to information indicating nutrients for which a user's intake is below predetermined reference values over a specified period.

The term “excess nutrient information” refers to information indicating nutrients for which a user's intake exceeds predetermined reference values over a specified period.

The term “prompt sentence” refers to a sequence of natural language tokens constructed to instruct a generative AI model, including context data, constraints, and an explicit request for generation of output content.

The term “output format condition information” refers to information specifying desired characteristics of generated output, including but not limited to length, structure, style, and level of detail.

The term “generative information processing model” refers to an information processing model, typically implemented by a neural network, that generates output data such as natural language text in response to input data including a prompt sentence.

The term “generation instruction information” refers to control information indicating that a particular prompt sentence is to be supplied as input to a generative information processing model and defining how the model is to be invoked.

The term “advice information” refers to information generated by a generative information processing model or by further processing thereof, the information including recommendations or guidance related to nutrition, health behavior, or similar topics.

The term “content correction processing” refers to processing that modifies advice information to correct, filter, or adjust content according to predefined rules, constraints, or additional context.

The term “expression adjustment processing” refers to processing that modifies wording, tone, or style of advice information to match user attributes, preferences, or situational context.

The term “user-oriented nutrient supplementation advice information” refers to advice information that has been corrected and adjusted for presentation to a specific user, the information including suggestions for nutrient intake or related behavior.

The term “interactive display unit” refers to a user interface component capable of displaying information and receiving user input in a dialog-like manner, including but not limited to a graphical user interface on a terminal.

The term “additional input information” refers to user-provided data submitted after initial advice is presented, including questions, feedback, preferences, or clarifications relevant to further advice generation.

The term “emotion information” refers to information related to an emotional state of a user, derived from or represented by at least one of voice data, image data, or text data.

The term “emotion estimation model” refers to a computational model configured to receive emotion information and output emotion state information, typically using pattern recognition or machine learning techniques.

The term “emotion state information” refers to information expressing an estimated or classified emotional state of a user, including but not limited to categories such as stress, fatigue, happiness, or neutrality, and associated intensities.

The term “emotion state summary information” refers to condensed emotion state information suitable for inclusion in a prompt sentence while capturing essential aspects of a user's emotional condition.

The term “output condition information corresponding to the emotion state” refers to information specifying how generated content should be adapted in view of a user's emotional state, such as adjusting strictness, tone, or focus.

The term “nutrient supplement information” refers to information recommending intake of additional nutrients, including but not limited to specific nutrient categories or nutrient-containing products.

The term “meal composition information” refers to information indicating combinations of food items forming a meal, designed to satisfy certain nutritional or contextual criteria.

The term “rule group” refers to a set of predefined conditions and associated actions used by a processor to modify or interpret advice information or related data.

The term “learned model” refers to a computational model trained using data to map input variables to output variables, used to adjust or generate information such as recommendations or dialog content.

The term “importance” refers to a relative degree of priority or emphasis assigned to individual recommendation elements within advice information.

The term “strictness” refers to a degree of constraint or rigidity in recommended behavior or guidance expressed in advice information.

The term “expression tone” refers to stylistic characteristics of language used in advice information, including but not limited to politeness level, directness, and emotional warmth.

The term “bidirectional dialog processing” refers to processing in which a system exchanges messages interactively with a user, receiving user inputs and providing system responses in alternating turns.

The term “voice dialog format” refers to a dialog interaction mode in which user inputs and system outputs are conveyed primarily as audio signals or speech.

The term “character dialog format” refers to a dialog interaction mode in which user inputs and system outputs are conveyed primarily as text characters displayed on a user interface.

The term “response information of the user” refers to data representing replies, questions, or feedback provided by a user during bidirectional dialog processing.

In one embodiment, a server cooperates with one or more terminals operated by a user to implement the claimed system. The server includes at least one processor, a memory storing executable programs and parameter data, and a storage device storing structured data such as user profiles, nutrient information, and historical records. The terminal includes at least one processor, a camera, one or more motion sensors (for example, an accelerometer and a gyroscope), a position information acquisition unit (for example, a satellite positioning receiver), a microphone, a display, and a wireless communication interface. The server and the terminal communicate via a packet-switched network using a secure transport protocol such as HTTPS.

The server executes an integrated program that performs multi-modal data processing, nutritional status evaluation, emotion estimation, prompt sentence construction, interaction with a generative AI model, and generation of user-oriented nutrient supplementation advice. The terminal executes a client-side program that captures images and sensor data, displays interactive dialogs, and transmits or receives structured messages to and from the server.

The server uses a relational database management system as a central data store. The database includes, for example, a user profile table, a meal image table, a meal ingredient table, a meal nutrient table, an exercise session table, an energy expenditure table, an emotion table, and a recommendation table. Each table has defined schema fields and foreign key relationships so that the server can associate, for each user, image-based meals, exercise sessions, energy expenditure values, emotion states, and generated advice.

The server uses a nutrition information storage unit implemented as one or more database tables that store nutrient data for food categories. Each record includes a food category identifier, a set of standardized nutrient attributes (for example, energy, protein, fat, carbohydrate, minerals, vitamins), and reference values per unit mass or per serving. The server associates detected ingredient information with these food categories to estimate intake nutrient information.

The terminal captures meal information by controlling the built-in camera. The terminal converts raw sensor frames into compressed image files, attaches metadata such as a user identifier, capture time, and context tags, and transmits the image information to the server. The server stores the image file in a non-volatile storage device and records a reference path and metadata in the meal image table. By storing only the reference path and associated keys in the database, the server reduces main database size and improves query performance when the number of users increases.

The server performs object recognition processing and feature extraction processing on meal image information using a convolutional neural network implemented in a machine learning framework such as a generic deep learning library. The server preprocesses each image by resizing it to a fixed input resolution, normalizing pixel values, and optionally applying color normalization. The server then passes the preprocessed image data as a tensor to a trained convolutional neural network that includes convolutional layers, batch normalization layers, non-linear activation functions, pooling layers, and fully connected layers. The network outputs posterior probabilities for a set of predefined food categories.

The server selects categories whose probabilities exceed a threshold and treats them as ingredient information.

The server estimates portion size by using either bounding box area ratios output by the convolutional neural network or a separate regression head that maps intermediate feature maps to estimated mass values. In an alternative embodiment, the server applies a secondary model that uses image features and historical consumption patterns of the same user to refine portion estimates. By including both classification and regression outputs, the server reduces the average error of predicted portions and thus improves nutrient estimation accuracy.

The server maps ingredient information to records in the nutrition information storage unit using category identifiers. For each detected ingredient, the server retrieves corresponding nutrient attributes and multiplies them by the estimated portion size. The server aggregates results for all ingredients in a meal and stores intake nutrient information in the meal nutrient table. The server simultaneously updates a daily aggregate table that accumulates nutrient totals and energy intake over rolling time windows.

The terminal acquires exercise information by reading outputs from motion sensors and the position information acquisition unit. The terminal samples acceleration and position at a predetermined rate and segments the time series into contiguous exercise sessions based on observed movement patterns. In one embodiment, the terminal uses a lightweight activity classification model that receives features such as mean acceleration magnitude, variance, step frequency, and GPS speed and outputs activity labels such as walking, running, or stationary. The terminal summarizes each session into a JSON-like structure including user identifier, start time, end time, activity type, distance, and, if available, heart rate statistics, and transmits this structure to the server.

The server stores exercise sessions in the exercise session table and computes activity intensity information and energy expenditure information. The server may employ a look-up table of metabolic equivalents for activity types and combine these with user attribute information such as body mass to compute energy expenditure. In an alternative embodiment, the server runs a regression model trained on sensor features and reference energy expenditure data to output more accurate energy values. By performing such calculations centrally and using standardized data structures, the server reduces device-dependent variation and improves proportional scaling when many users are served concurrently.

The server integrates intake nutrient information, energy expenditure information, user attribute information, and health-related information over a predetermined period to generate nutritional status evaluation information. The server retrieves daily intake totals, daily energy expenditure figures, and any available health-related data such as sleep duration or self-reported symptoms from the database. The server then computes nutrient sufficiency information by dividing the intake of each nutrient by reference values stored in a reference table. The server also computes energy balance information by subtracting energy expenditure from energy intake over the same period. The server stores these results in a normalized form as nutritional status evaluation information, which allows efficient subsequent retrieval for prompt sentence construction and statistical analysis.

The server acquires emotion information of the user as at least one of voice information, image information, and character string information. The terminal may capture voice signals via the microphone, convert them into digital samples, and transmit them to the server. The server extracts acoustic features such as pitch, energy, spectral centroids, and Mel-frequency cepstral coefficients and inputs these features into an emotion estimation model. The emotion estimation model can be a recurrent neural network or a transformer-based network that has been trained on labelled emotional speech data using a supervised learning procedure with a cross-entropy loss function. The server updates network weights during training by using gradient descent with backpropagation. When the terminal transmits image information such as a user face, the server pre-processes the image to detect the face region, extracts facial landmarks, and calculates features such as muscle movement intensities or action unit activations. These features are input to a neural network that outputs an emotion classification or a continuous arousal-valence representation. When the terminal transmits character string information such as text messages, the server tokenizes the text, applies an embedding layer, and passes the embedded sequence into a text classifier that has been supervised to predict emotion categories. In all cases, the server converts intermediate outputs of these models into emotion state information such as “high stress,” “fatigue,” or “positive mood” and stores them along with timestamps in the emotion table.

The server generates evaluation summary information by selecting key aspects of nutritional status evaluation information and emotion state information. For example, the server identifies nutrient categories whose sufficiency ratios fall below a first threshold as deficiencies and those above a second threshold as excesses, and compiles deficiency nutrient information and excess nutrient information. The server also reduces a potentially large number of emotion records into an emotion state summary, such as a dominant emotion over the last day. The server then constructs a prompt sentence for a generative AI model by concatenating textual components according to a structured template. The template includes segments representing energy balance, major nutrient deficiencies, relevant emotion state, and explicit instructions regarding output format and content scope. As a specific example, the server may construct the following prompt sentence:

“Based on the following user data, evaluate nutritional status and generate practical advice.
Average energy intake: 2100 kcal
Average energy expenditure: 2400 kcal
Vitamin B1 intake: 60% of recommended
Vitamin C intake: 50% of recommended
Emotion: user feels tired and stressed.

Please recommend specific foods and supplements to improve vitamin B1 and vitamin C intake, and give short explanations on how they may help fatigue and stress. Answer in less than 300 words.”

The server supplies the constructed prompt sentence as input to a generative AI model, which may be an autoregressive language model implemented as a transformer network. The model includes multiple layers of self-attention mechanisms, feed-forward layers, and layer normalization. During prior training, the model has been optimized on large corpora of text by minimizing a cross-entropy loss between predicted and actual tokens, with parameters updated using stochastic gradient descent. In some embodiments, the server fine-tunes the model on domain-specific dialogs about nutrition and health, using user questions and curated responses as training examples.

The server interacts with the generative AI model through an application programming interface. The server encapsulates the prompt sentence, optionally along with system-level directives (for example, constraints against medical diagnoses), in a request message. The server transmits this message to the model host and receives generated advice information as a response. The generated advice information typically includes natural language sentences that recommend foods, highlight nutrient deficiencies, or suggest simple behavioral changes.

The server performs content correction processing and expression adjustment processing on the received advice information. The server may apply a rule group that filters prohibited expressions, removes ambiguous terms, or enforces inclusion of safety disclaimers. The server may further adjust the expression tone by using a smaller neural model that predicts user preference for directness or politeness based on user attribute information and prior feedback. The server replaces, deletes, or reformulates parts of the advice text to accommodate such preferences. For example, when the emotion state information indicates high stress, the server may reduce imperative wording and insert more supportive language.

The server generates user-oriented nutrient supplementation advice information by combining corrected advice text with structured references to actual food categories and nutrient metrics from the database. In one embodiment, the server includes clearly linked identifiers so that the terminal can highlight recommended foods and show nutrient breakdown in a graphical interface. The server writes this user-oriented advice information to the recommendation table and transmits a copy to the terminal.

The terminal displays the user-oriented nutrient supplementation advice information in an interactive display unit configured as a chat-like interface. The terminal renders the advice as time-ordered messages, allowing the user to scroll through past content. The terminal also provides input controls for the user to enter additional questions or clarifications as free text or voice input. The terminal encodes new user messages as additional input information and transmits them to the server.

The server appends the additional input information to the conversation history stored for that user. The server generates a new prompt sentence that maintains conversation context by including a short summary of previous system advice and an explicit indication of the new user query. For example, the server may construct a prompt sentence such as:

“The user previously received advice to increase vitamin B1 and vitamin C intake by consuming whole grains, pork, green vegetables, and citrus fruits.

The user now asks: ‘Are there convenient snacks rich in vitamin B1 that can be bought at a convenience store?’

Please answer with three concrete snack examples and briefly explain why they are rich in vitamin B1.”

By automatically generating such context-rich prompt sentences and iteratively invoking the generative AI model, the server enables a bidirectional dialog that remains coherent across turns. The server also logs user responses as component information for subsequent prompt sentences, so that the system learns which types of suggestions are accepted or rejected and can adjust future advice generation accordingly.

The described architecture improves computer technology in several ways. First, the server centralizes integration of heterogeneous data sources (image information, sensor streams, text, and audio) into normalized representations and reuses these representations for nutritional evaluation and prompt construction, thereby reducing redundant parsing and serialization operations that would otherwise occur if each service processed raw data independently. Second, by building prompt sentences from structured evaluation summary information rather than raw logs, the server reduces prompt length and concentrates information content, which decreases network bandwidth to the generative AI model host and shortens model inference time. Third, the server applies algorithmic rules to select only those nutrient categories and emotion states that satisfy threshold conditions, which narrows the search space of relevant recommendations and leads to more stable and efficient generation.

Furthermore, the server improves accuracy of both nutrient estimation and emotional adaptation by designing specific neural network architectures for object recognition, portion estimation, and emotion classification. These models consume feature sets not typically used in manual pipelines, such as dense image embeddings, time-domain and frequency-domain acoustic features, and multi-turn conversation encodings. These features enable the server to infer subtle patterns in consumption and mood that are not readily captured by human operators or conventional rule-based systems. Because the server uses learned models with differentiable parameters and trains them using explicit loss functions, the system can continuously reduce prediction error as more training data is accumulated.

The system also reduces communication load by allowing the terminal to perform preliminary segmentation and classification of exercise sessions and by transmitting aggregated session descriptors rather than raw sensor time series. This design reduces the volume of data per user while preserving sufficient information for accurate energy expenditure computation, thereby improving scalability of the server. In alternative embodiments, the terminal may also perform preliminary face detection or voice activity detection to further decrease data size before transmission.

In another embodiment, the server executes the generative AI model locally using graphics processing units or dedicated accelerators. In such a configuration, the server caches embeddings or intermediate states for frequently used prompt templates and user contexts, so that subsequent prompt sentences can be processed faster by reusing precomputed attention keys and values. This mechanism improves inference throughput and reduces average response latency.

The system is not limited to a single generative AI model. In yet another embodiment, the server uses a first model specialized in nutritional text and a second model specialized in conversational tone adaptation. The server uses a deterministic algorithm to partition the prompt sentence into a factual part sent to the first model and a stylistic part processed by the second model. The server then merges the outputs using a rule group that prioritizes factual correctness and consistent tone. This modular design enhances robustness and allows independent updating of each model without requiring changes to the overall architecture.

In all embodiments, the server, the terminal, and the user cooperate through clearly defined data structures and processing modules. The server's use of prompt sentences derived from structured evaluation summary information and emotion state information, combined with learned neural network models and rule-based post-processing, provides a specific, technical way to generate adaptive, context-aware advice that cannot be straightforwardly replicated by manual workflows or simple automation. As a result, the system yields technical effects such as faster response times, improved accuracy of nutrient and emotion estimation, reduced communication bandwidth, and more coherent long-term dialog, thereby representing an improvement to computer-implemented processing rather than a mere automation of human nutrition counseling.

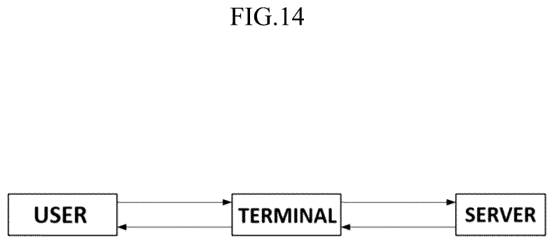

The following describes the processing flow using FIG. 14.

### Step 1:

User operates the terminal to capture a meal image using a built-in camera. The input is a real-world meal scene; the terminal converts this scene into digital image data (for example, a JPEG file) and attaches metadata such as user identifier, timestamp, and meal context (for example, “breakfast,” “restaurant”). The output is structured image information including the encoded image and associated metadata stored temporarily in the terminal's memory.

### Step 2:

Terminal transmits the structured image information to the server via a secure communication protocol. The input is the image file and metadata from Step 1; the terminal encapsulates these data into an HTTP request, performs encryption using a transport layer security protocol, and sends the request to a predefined server endpoint. The output is a network message received by the server that contains the meal image data and metadata.

### Step 3:

Server stores the received meal image and metadata in persistent storage. The input is the network message from Step 2; the server decodes the message, checks authentication tokens, validates the metadata format, and writes the image file to a non-volatile storage device while inserting a record into a meal image table with fields such as user identifier, storage path, and capture time. The output is a stored meal image reference that can be retrieved by key from the database.

### Step 4:

Server performs preprocessing on the stored meal image to generate a standardized input for a recognition model. The input is the raw meal image data retrieved from storage; the server uses an image processing library to resize the image to a fixed resolution, normalize pixel values, optionally apply color normalization and noise reduction, and convert the image into a multi-dimensional tensor. The output is a preprocessed image tensor suitable for input to a convolutional neural network.

### Step 5:

Server executes object recognition processing and feature extraction on the preprocessed image tensor to generate ingredient information. The input is the preprocessed tensor from Step 4; the server feeds this tensor to a convolutional neural network that includes convolutional layers, pooling layers, and fully connected layers, computes forward passes to obtain class probabilities for predefined food categories, and extracts intermediate feature maps. The output is ingredient information including a list of detected food categories with associated confidence scores and optional spatial or portion indicators.

### Step 6:

Server estimates portion sizes for detected ingredients and computes intake nutrient information. The input is the ingredient information from Step 5 and mass or serving estimation parameters stored in the server; the server applies regression functions or learned mappings to convert bounding box sizes or feature vectors into estimated masses or serving counts, and multiplies nutrient values per unit from a nutrient information storage unit by these masses. The output is intake nutrient information describing quantities of energy and multiple nutrients per ingredient and aggregated per meal.

### Step 7:

Terminal acquires exercise information using motion sensors and a position information acquisition unit. The input is raw sensor readings such as acceleration vectors, angular velocities, and position coordinates sampled over time while the user performs physical activity; the terminal segments the time series into candidate activity periods, calculates features such as mean acceleration, variance, step frequency, and distance, and labels each segment with an activity type if a lightweight classifier is present. The output is exercise session data including user identifier, start time, end time, activity type, distance, and summary features.

### Step 8:

Terminal transmits exercise session data to the server for central processing. The input is the session data from Step 7; the terminal serializes the data into a structured format, attaches authentication information, and sends it via a secure communication protocol. The output is a received data packet at the server that encodes the user's exercise sessions and related features.

### Step 9:

Server computes activity intensity information and energy expenditure information from the received exercise session data. The input is the exercise session data from Step 8 and user attribute information such as body mass and age retrieved from the database; the server applies metabolic equivalent values or a regression model to the session features, multiplies by body mass and duration to obtain energy expenditure, and quantizes intensity levels into categories (for example, low, moderate, high). The output is activity intensity information and energy expenditure information recorded in exercise-related tables.

### Step 10:

Server integrates intake nutrient information, energy expenditure information, user attribute information, and other health-related information to generate nutritional status evaluation information. The input is meal nutrient records from Steps 5 and 6, energy expenditure records from Step 9, and long-term health-related data such as sleep duration or self-reported conditions; the server sums nutrient intakes over a predetermined time window, sums energy expenditure over the same window, compares nutrient intakes to reference values, and computes ratios and differences representing sufficiency and energy balance. The output is nutritional status evaluation information including nutrient sufficiency information and energy balance information for the user.

### Step 11:

Terminal or user application provides emotion information to the server as voice, image, or text data. The input is user voice signals collected by a microphone, face images captured by a camera, or text messages typed or selected by the user; the terminal converts these into digital formats, adds timestamps and user identifiers, and transmits them to the server. The output is emotion input data stored or buffered at the server for emotion analysis.

### Step 12:

Server applies an emotion estimation model to the emotion input data and generates emotion state information. The input is the emotion input data from Step 11; the server extracts features such as acoustic descriptors from voice, facial landmarks from images, or token embeddings from text, and passes these feature vectors into one or more trained neural networks or classifiers that output probabilities over emotion categories or continuous arousal-valence values. The output is emotion state information indicating one or more emotion categories and intensities, which the server writes into an emotion table.

### Step 13:

Server generates evaluation summary information by combining nutritional status evaluation information and emotion state information. The input is nutritional status evaluation information from Step 10 and emotion state information from Step 12; the server selects nutrients with sufficiency ratios below or above predefined thresholds to determine deficiency nutrient information and excess nutrient information, computes representative energy balance metrics, and reduces multiple emotion records to a dominant or aggregate emotion state over the evaluation window. The output is evaluation summary information that condenses nutritional, energetic, and emotional status into a compact representation.

### Step 14:

Server constructs a prompt sentence for a generative AI model based on the evaluation summary information and output format requirements. The input is the evaluation summary information from Step 13 and stored prompt templates or rules; the server fills template slots with current values such as energy intake, energy expenditure, deficient nutrients, excess nutrients, and emotion summaries, and appends explicit instructions regarding desired output length, structure, and focus. The output is a natural-language prompt sentence that encodes the user's condition and a specific generation task for the generative AI model.

### Step 15:

Server transmits the constructed prompt sentence to the generative AI model and obtains advice information. The input is the prompt sentence from Step 14; the server wraps the sentence and optional system instructions into a model request, sends the request to a model hosting environment, and waits for completion of sequence generation using a transformer-based architecture that predicts subsequent tokens conditioned on the prompt.

The output is advice information in the form of generated natural-language text describing recommended foods, nutrients, and actions.

### Step 16:

Server performs content correction processing and expression adjustment processing on the advice information to generate user-oriented nutrient supplementation advice information. The input is the generated advice information from Step 15, user attribute information, and emotion state information; the server applies rule sets to filter or correct content (for example, removing unsuitable recommendations for known allergies), and may pass the advice through an additional model or rule group to soften or strengthen tone based on user preferences and emotional state. The output is user-oriented nutrient supplementation advice information tailored to the current user.

### Step 17:

Server records the user-oriented nutrient supplementation advice information and transmits it to the terminal for presentation. The input is the tailored advice from Step 16; the server stores the advice along with metadata in a recommendation table to maintain history and then packages the advice into a response message to the terminal. The output is a transmitted payload received by the terminal that contains the final advice text and optional structured references.

### Step 18:

Terminal presents the user-oriented nutrient supplementation advice information via an interactive display unit and acquires additional input information from the user. The input is the advice payload from Step 17; the terminal renders the advice as chat-style messages, may highlight key items or nutrient values, and waits for user interactions such as scrolling, tapping, or entering follow-up questions. The output is additional input information, including new user questions or feedback, captured as text or voice and prepared for sending to the server.

### Step 19:

Server incorporates the additional input information into a new prompt sentence and performs iterative input to the generative AI model for continuous update of advice. The input is the additional input information from Step 18 and the previous conversation history stored in the database; the server updates the conversation context, summarizes prior system responses and user reactions, and constructs a new prompt sentence that includes the updated context and the new user request. The server then repeats the interaction with the generative AI model as in Steps 15 and 16, generating updated advice information that reflects both prior recommendations and newly expressed user needs. The output is refined user-oriented nutrient supplementation advice information that continues the dialog and is suitable for further presentation and interaction.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naive Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

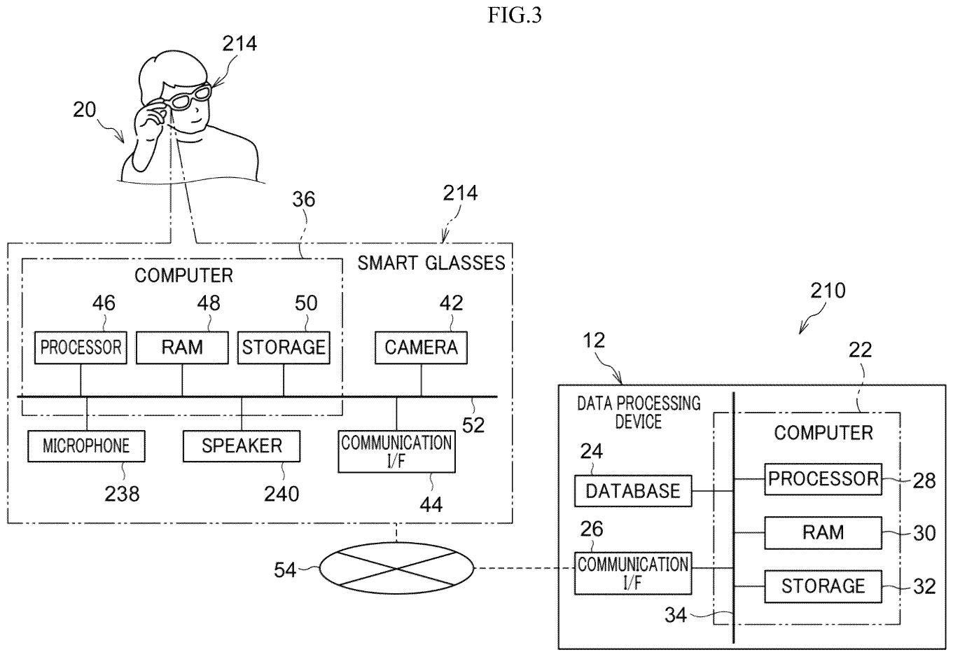

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

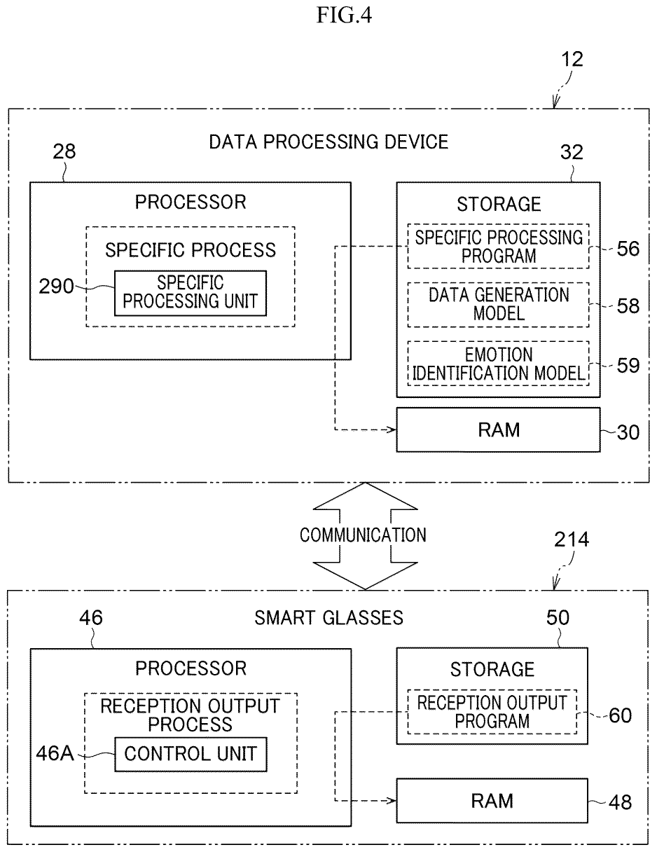

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naive Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

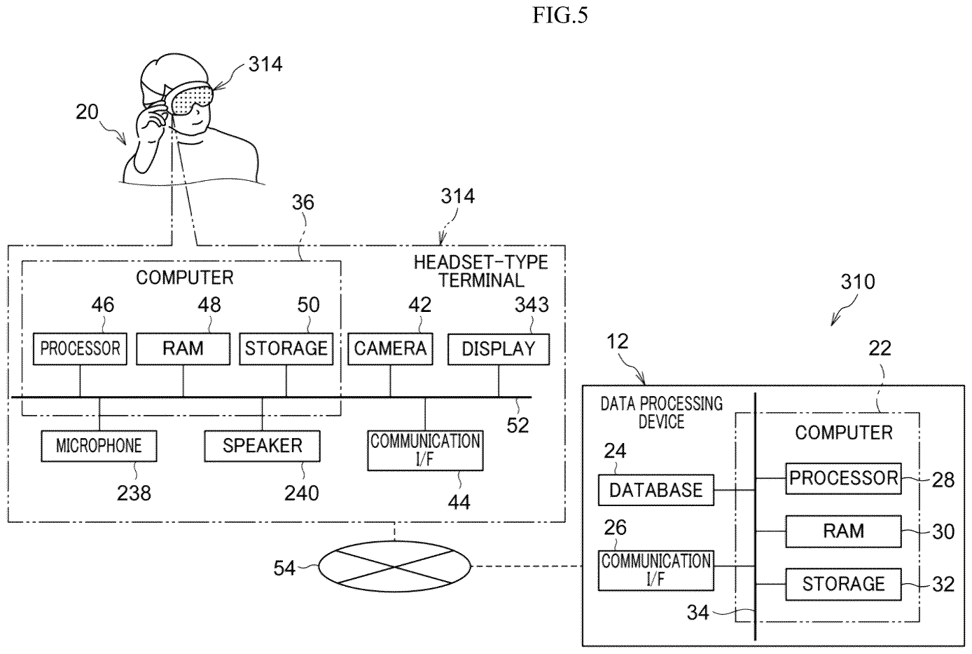

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

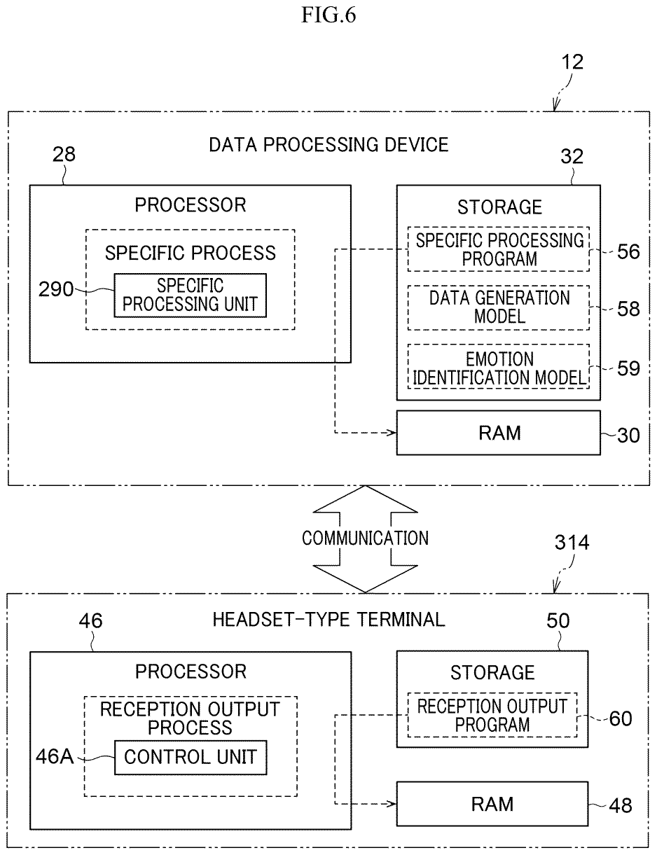

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naive Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

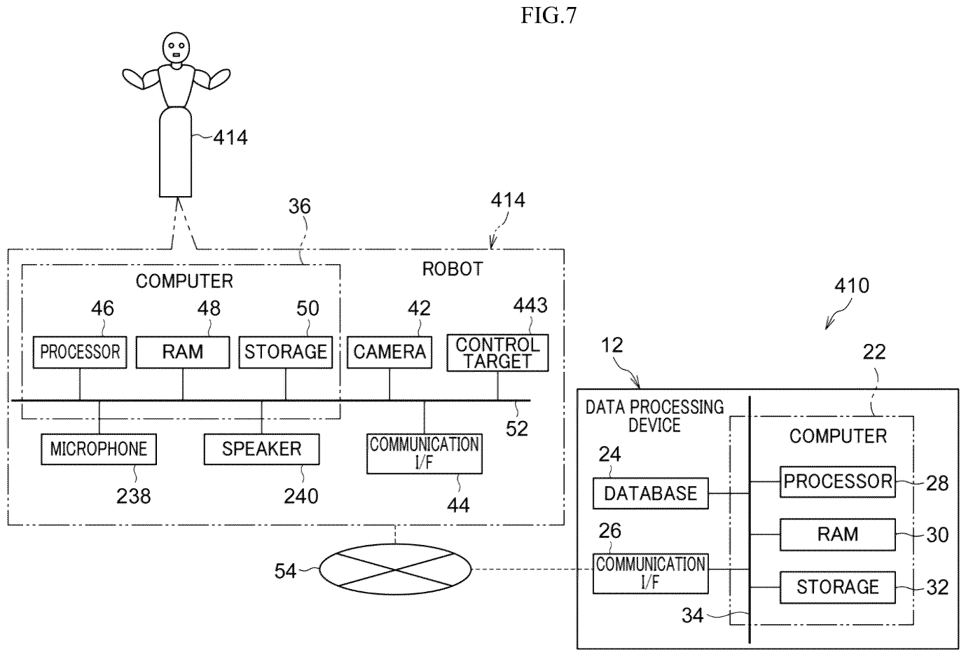

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

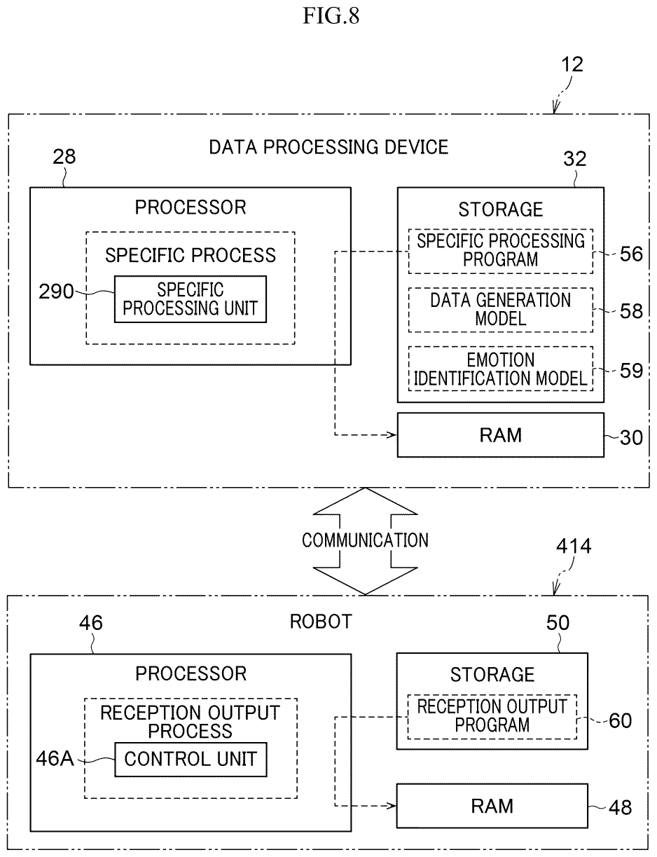

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

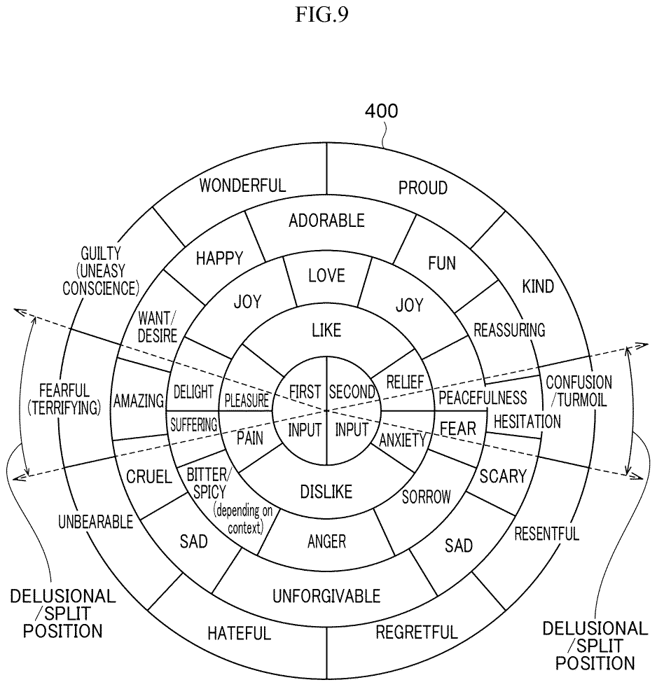

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

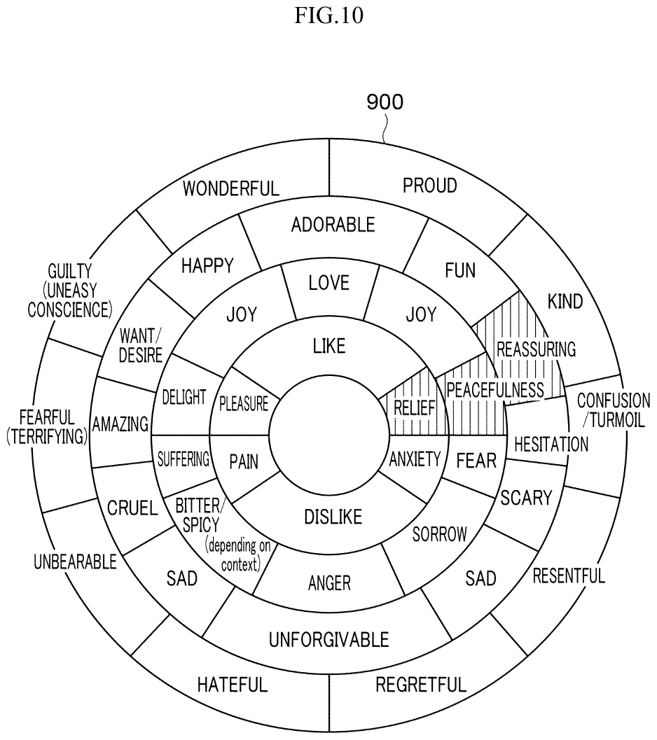

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (Saas).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor,
wherein the processor is configured to
acquire meal information of a user as image information and character information, analyze the image information to classify intake objects, and calculate nutrition-related information on the basis of a classification result of the intake objects and the character information,
acquire exercise amount information of the user as time-series data by using a motion detection device and a position specifying technique, calculate a movement distance, a step count, and an energy consumption on the basis of the time-series data, and store exercise record information,
store the nutrition-related information and the exercise record information in a storage device, aggregate a nutrition intake amount and an exercise amount over a predetermined period, and generate a health status index,
construct a prompt sentence to be used as an input to a generative AI model on the basis of the health status index and attribute information of the user, include the nutrition-related information and the exercise record information in the prompt sentence, and transmit the prompt sentence to the generative AI model,
analyze evaluation information and improvement proposal information in natural language obtained from the generative AI model, and organize and store the evaluation information and the improvement proposal information as a health evaluation result and behavioral guideline information for each user, and
deliver the health evaluation result and the behavioral guideline information to a terminal device and present the health evaluation result and the behavioral guideline information to the user by display control.

### (Supplementary 2)

The system according to supplementary 1,
wherein the processor is configured to dynamically generate the prompt sentence on the basis of a nutrition deficiency state of the user, emotion state information, and past accumulated records, and include in the prompt sentence an instruction to cause the generative AI model to generate recommendations of specific nutritional supplements, intake plans, and exercise plans.

### (Supplementary 3)

The system according to supplementary 1,
wherein the processor is configured to convert the evaluation information and the improvement proposal information into a dialogue-type response sentence by using a language processing function, provide the dialogue-type response sentence to the user as a sequential question-and-answer display on the terminal device, and transmit additional input content from the user as an additional prompt sentence to the generative AI model.

### Application Example 1

### (Supplementary 1)

A system comprising a processor,
wherein the processor is configured to
control an imaging device provided in a user terminal to acquire image data representing ingested food and beverage of a user, execute image analysis processing based on machine learning on the image data to identify a type and an amount of the food and beverage, and refer to a nutrition information storage unit in which nutrition information is associated with the type and the amount of the food and beverage to calculate nutrient component amounts of the food and beverage,
control an acceleration detection device and a location information acquisition device provided in the user terminal to acquire time-series sensor data and movement route data relating to physical activity of the user, and calculate an activity type, a movement distance, a speed, and an energy expenditure amount based on the acquired data, aggregate an intake energy amount and the energy expenditure amount for a predetermined period based on the calculated nutrient component amounts and the calculated energy expenditure amount, estimate a basal metabolism amount and a target energy balance using user profile information, evaluate a nutritional status of the user by comparison with the basal metabolism amount and the target energy balance, and generate structured evaluation data representing an evaluation result,
summarize, in a natural language, the structured evaluation data and a dietary history data and a physical activity history data for the predetermined period, generate a text including an age, a body constitution, a target, and the evaluation result of the user, and construct a prompt sentence for input to a generative artificial intelligence model by adding to the text instructions relating to an output format and recommended content,
transmit the prompt sentence to the generative artificial intelligence model, acquire an advice text relating to nutrition supplementation generated by the generative artificial intelligence model, store the advice text in an advice history storage unit in association with the user, and transmit the advice text to the user terminal.

### (Supplementary 2)

The system according to supplementary 1,
wherein the processor is configured to
estimate an emotional state of the user based on information relating to an emotional index acquired from the user terminal and self-report information of the user, generate a prompt sentence that integrates, in a natural language, the emotional state and the structured evaluation data generated in the system according to supplementary 1, and include, in the prompt sentence, a text that instructs the generative artificial intelligence model to generate a recommendation of a specific nutritional supplement or a meal menu.

### (Supplementary 3)

The system according to supplementary 1,
wherein the processor is configured to
store, in association with dialogue history data, the advice text generated by the generative artificial intelligence model in the system according to supplementary 1, generate, as a new prompt sentence, a combination of an additional input text from the user with the dialogue history data and the structured evaluation data, input the new prompt sentence to the generative artificial intelligence model, acquire a response text from the generative artificial intelligence model, and sequentially transmit the response text to the user terminal to present continuous advice in a dialogue format using natural language processing.

### Example 2

### (Supplementary 1)

A system comprising a processor,
wherein the processor is configured to
acquire meal image data and physical activity data transmitted from a user terminal via a communication network, and store the meal image data and the physical activity data in a storage device,
transmit the meal image data to an external processing resource or externally provided function for image recognition, identify food components based on an identification result acquired from the external processing resource or the externally provided function, and associate the food components with standardized food information,
acquire nutrition information corresponding to the standardized food information from a nutrition information storage unit, and calculate an intake nutrient amount for each meal or for a predetermined period based on the acquired nutrition information and an estimated intake amount of the food components,
refer to metabolic equivalent information based on an exercise type, an exercise time, and body information included in the physical activity data, and calculate an energy expenditure amount for the predetermined period by using the metabolic equivalent information,
generate nutrition state evaluation information indicating an energy balance and a surplus or deficiency of each nutrient by comparing the intake nutrient amount and the energy expenditure amount with predetermined reference information and user attribute information,
construct a prompt sentence for instructing a generative AI model to generate a nutrition support proposal and an eating habit improvement proposal based on the food components, the intake nutrient amount, the energy expenditure amount, the nutrition state evaluation information, and goal information of a user, and transmit the prompt sentence to the generative AI model, and
format proposal information acquired from the generative AI model as guidance information in a natural language for the user, and notify the guidance information to the user terminal.

### (Supplementary 2)

The system according to supplementary 1,
wherein the processor is configured to
dynamically change an expression style, a level of detail, and a priority of recommended content included in the prompt sentence based on psychological information of the user, behavior history information of the user, and the nutrition state evaluation information, so that the proposal information generated by the generative AI model is adapted to goals and preferences of the user.

### (Supplementary 3)

The system according to supplementary 1,
wherein the processor is configured to
include dialogue history information and response format specification information in the prompt sentence in order to cause the guidance information to be generated as conversational responses, and to sequentially transmit and receive the conversational responses between the system and the user terminal through dialogue management processing based on conversational responses acquired from the generative AI model.

### Application Example 2

### (Supplementary 1)

A system comprising a processor,
wherein the processor is configured to
acquire meal information of a user as image information from a terminal, perform object recognition processing and feature extraction processing on the image information to specify ingredient information, and estimate intake nutrient information by referring to a nutrient information storage unit associated with the ingredient information,
acquire exercise information of the user from at least one motion sensor and at least one position information acquisition unit, and calculate activity intensity information and energy expenditure information based on the exercise information,
integrate the intake nutrient information and the energy expenditure information with user attribute information and health-related information over a predetermined period, and generate nutritional status evaluation information of the user by calculating nutrient sufficiency information and energy balance information relative to predetermined reference information,
generate evaluation summary information including at least deficiency nutrient information and excess nutrient information from the nutritional status evaluation information and the health-related information, construct a prompt sentence including the evaluation summary information and output format condition information, and generate generation instruction information that instructs input of the prompt sentence to a generative information processing model, acquire advice information output from the generative information processing model,
perform content correction processing and expression adjustment processing on the advice information based on the user attribute information and the health-related information, and generate user-oriented nutrient supplementation advice information, and
present the user-oriented nutrient supplementation advice information in time series via an interactive display unit, acquire additional input information from the user, generate a new prompt sentence including the additional input information, and perform iterative input to the generative information processing model to execute continuous update of advice.

### (Supplementary 2)

The system according to supplementary 1,
wherein the processor is configured to
acquire emotion information of the user as at least one of voice information, image information, and character string information, apply an emotion estimation model to the emotion information to calculate emotion state information, associate the emotion state information with the nutritional status evaluation information, dynamically change configuration content of the prompt sentence so as to include emotion state summary information and output condition information corresponding to the emotion state in the prompt sentence, and instruct the generative information processing model to present nutrient supplement information or meal composition information adapted to the emotion state.

### (Supplementary 3)

The system according to supplementary 1,
wherein the processor is configured to
apply a rule group or a learned model based on the nutritional status evaluation information of the user and the emotion state information to the advice information acquired from the generative information processing model, adjust importance, strictness, and expression tone of recommended contents, execute bidirectional dialog processing with the user in a voice dialog format or a character dialog format using adjusted advice information, and record response information of the user in the bidirectional dialog processing as component information of the prompt sentence for subsequent processing.

What is claimed is:

1. A system comprising:
circuitry configured to:
receive, via a communication interface coupled to a packet-switched network, image data and time-series sensor data from a terminal device;
transmit the image data to an object recognition resource, acquire identification result data from the object recognition resource, and associate the identification result data with reference entries stored in a database to generate component data;
compute a first numerical parameter set based on the component data and quantity estimation data derived from the identification result data;
compute a second numerical parameter set based on the time-series sensor data and position data acquired from a location information acquisition device of the terminal device;
generate structured evaluation data by aggregating the first numerical parameter set and the second numerical parameter set over a predetermined period, comparing aggregated values with baseline reference data and target parameter data derived from attribute data of a user, and producing surplus-or-deficiency indicator data;
construct a prompt data structure comprising a natural-language summary of the structured evaluation data, the attribute data, and instruction data specifying an output format and a content category, and transmit the prompt data structure to a generative neural network model via the packet-switched network; and
acquire generated text data from the generative neural network model, store the generated text data in a history storage device in association with the user, and transmit the generated text data to the terminal device via the communication interface.

2. The system according to claim 1, wherein the circuitry is configured to compute the first numerical parameter set by referencing a structured information storage that maps each reference entry to a plurality of quantitative attribute values comprising at least an energy value and a plurality of component ratios.

3. The system according to claim 2, wherein the circuitry is configured to compute the second numerical parameter set by referencing intensity coefficient data associated with a detected activity classification, a duration value, and a body-related parameter of the user to produce an energy expenditure value.

4. The system according to claim 3, wherein the component data comprises food ingredient data identified by the object recognition resource from a meal photograph, and wherein the first numerical parameter set comprises intake nutrient amounts including at least a calorie value, a protein amount, a fat amount, and a carbohydrate amount.

5. The system according to claim 4, wherein the circuitry is configured to generate the structured evaluation data by calculating a net energy balance from the intake nutrient amounts and the energy expenditure value, determining a surplus or deficiency for each of the protein amount, the fat amount, and the carbohydrate amount relative to recommended reference values, and encoding the surplus or deficiency as the surplus-or-deficiency indicator data.

6. The system according to claim 5, wherein the instruction data in the prompt data structure specifies that the generative neural network model generate a nutritional supplement recommendation and a meal menu recommendation based on the surplus-or-deficiency indicator data.

7. The system according to claim 1, wherein the circuitry is configured to construct the prompt data structure by selecting, from the history storage device, dialogue history data comprising prior prompt data structures and prior generated text data associated with the user, and embedding the dialogue history data as context within the prompt data structure.

8. The system according to claim 7, wherein the circuitry is configured to receive additional input data from the terminal device, generate a follow-up prompt data structure by combining the additional input data with the dialogue history data and the structured evaluation data, transmit the follow-up prompt data structure to the generative neural network model, and acquire a follow-up generated text data to present continuous guidance in a multi-turn dialogue format.

9. The system according to claim 1, wherein the circuitry is configured to estimate an emotional state of the user by applying an emotion estimation model to at least one of voice data, facial image data, or self-report text data received from the terminal device, and incorporate emotion state data into the prompt data structure as a conditioning parameter that modifies an expression style and a priority of content in the generated text data.

10. The system according to claim 9, wherein the circuitry is configured to dynamically adjust the instruction data within the prompt data structure based on the emotion state data by selecting from a plurality of expression templates, each expression template specifying a tone, a level of detail, and a maximum number of recommendation items.

11. The system according to claim 1, wherein the object recognition resource comprises a convolutional neural network trained to classify objects depicted in the image data and to output confidence scores for each classification, and wherein the circuitry is configured to filter the identification result data by retaining only classifications having confidence scores exceeding a predetermined threshold.

12. The system according to claim 11, wherein the circuitry is configured to perform preprocessing on the image data prior to transmission to the object recognition resource, the preprocessing comprising downsampling, format conversion, and metadata attachment indicating an acquisition timestamp and a category label.

13. The system according to claim 1, wherein the time-series sensor data comprises acceleration data sampled along a plurality of axes from an accelerometer of the terminal device, and wherein the circuitry is configured to apply a noise filtering algorithm and a step detection algorithm to the acceleration data to compute a step count and a movement distance.

14. The system according to claim 13, wherein the circuitry is configured to classify the time-series sensor data into a plurality of activity types by applying a classification model to feature vectors extracted from the acceleration data and the position data, the activity types comprising at least a walking type, a running type, and a resting type.

15. The system according to claim 1, wherein the circuitry is configured to generate the natural-language summary by inserting numerical values from the structured evaluation data into sentence template data stored in the database, the sentence template data comprising parameterized text segments specifying positions for the aggregated values and the surplus-or-deficiency indicator data.

16. The system according to claim 1, wherein the circuitry is configured to apply post-processing to the generated text data by performing rule-based filtering to remove inconsistent statements, segmenting the generated text data into a plurality of structured sections, and formatting each structured section for display on the terminal device.

17. The system according to claim 1, wherein the circuitry is configured to maintain user preference data comprising sensitivity parameters and behavior history data in the database, and to encode the user preference data into the instruction data of the prompt data structure to control a verbosity level and a recommendation priority of the generated text data.

18. A system comprising:
circuitry configured to:
receive, via a communication interface coupled to a packet-switched network, image data and sensor data from a terminal device;
transmit the image data to an object recognition resource and acquire identification result data comprising classification labels and confidence scores;
compute intake parameter data by associating the identification result data with reference entries in a structured information storage and aggregating quantitative attribute values corresponding to the reference entries;
compute expenditure parameter data by applying intensity coefficient data to the sensor data and position data received from the terminal device;
generate structured evaluation data by comparing the intake parameter data and the expenditure parameter data with baseline reference data derived from user attribute data, and producing indicator data representing a surplus or deficiency of each of a plurality of tracked parameters;
construct a prompt data structure by generating a natural-language summary of the structured evaluation data and appending instruction data specifying an output format, transmit the prompt data structure to a generative neural network model, and acquire generated guidance data from the generative neural network model; and
store the generated guidance data in a history storage device and transmit the generated guidance data to the terminal device via the communication interface.

19. The system according to claim 18, wherein the circuitry is configured to estimate an emotional state of a user associated with the terminal device by applying an emotion estimation model to at least one of voice data or text data received from the terminal device, and to modify the instruction data in the prompt data structure based on the emotional state to adjust an expression style and a content priority of the generated guidance data.

20. A method performed by circuitry, the method comprising:
receiving, via a communication interface coupled to a packet-switched network, image data and time-series sensor data from a terminal device;
transmitting the image data to an object recognition resource and acquiring identification result data from the object recognition resource;
computing a first numerical parameter set based on component data derived from the identification result data and reference entries stored in a database;
computing a second numerical parameter set based on the time-series sensor data and position data acquired from a location information acquisition device of the terminal device;
generating structured evaluation data by aggregating the first numerical parameter set and the second numerical parameter set over a predetermined period, comparing aggregated values with baseline reference data and target parameter data, and producing surplus-or-deficiency indicator data;
constructing a prompt data structure comprising a natural-language summary of the structured evaluation data and instruction data specifying an output format, and transmitting the prompt data structure to a generative neural network model via the packet-switched network; and
acquiring generated text data from the generative neural network model, storing the generated text data in a history storage device, and transmitting the generated text data to the terminal device via the communication interface.
